SEQUENCE LISTING (25 sequences)

SEQ ID NO: 1 - protein, 885 aa, Arabidopsis thaliana
MDLEANCKEKLSYFRIKELKDVLTQLGLSKQGKKQELVDRILTLLSDEQAARLLSKKNTVAKEAVAKLVDDTYRKMQVSGASDLASKGQVSSDTSNLKVKGEPEDPFQPEIKVRCVCGNSLETDSMIQCEDPRCHVWQHVGCVILPDKPMDGNPPLPESFYCEICRLTRADPFWVTVAHPLSPVRLTATTIPNDGASTMQSVERTFQITRADKDLLAKPEYDVQAWCMLLNDKVLFRMQWPQYADLQVNGVPVRAINRPGGQLLGVNGRDDGPIITSCIRDGVNRISLSGGDVRIFCFGVRLVKRRTLQQVLNLIPEEGKGETFEDALARVRRCIGGGGGDDNADSDSDIEVVADFFGVNLRCPMSGSRIKVAGRFLPCVHMGCFDLDVFVELNQRSRKWQCPICLKNYSVEHVIVDPYFNRITSKMKHCDEEVTEIEVKPDGSWRVKFKRESERRELGELSQWHAPDGSLCPSAVDIKRKMEMLPVKQEGYSDGPAPLKLGIRKNRNGIWEVSKPNTNGLSSSNRQEKVGYQEKNIIPMSSSATGSGRDGDDASVNQDAIGTFDFVANGMELDSISMNVDSGYNFPDRNQSGEGGNNEVIVLSDSDDENDLVITPGPAYSGCQTDGGLTFPLNPPGIINSYNEDPHSIAGGSSGLGLFNDDDEFDTPLWSFPSETPEAPGFQLFRSDADVSGGLVGLHHHSPLNCSPEINGGYTMAPETSMASVPVVPGSTGRSEANDGLVDNPLAFGRDDPSLQIFLPTKPDASAQSGFKNQADMSNGLRSEDWISLRLGDSASGNHGDPATTNGINSSHQMSTREGSMDTTTETASLLLGMNDSRQDKAKKQRSDNPFSFPRQKRSVRPRMYLSIDSDSETMNRIIRQDTGV

SEQ ID NO: 2 - protein, 888 aa, Medicago truncatula
MDDLVSSCKEKLQYFRVKDLKDVLTQIGISKQGKKQDLIDRILSIISDEQVAKVRAKKNAVEKEQVVKLVEDTYRKLQVSGATDIASKGQVASDSSNVKIKGEVEDSVQSATKVRCLCGSSLETDLLIKCEDRKCPVSQHLNCVIIPDTPTEGLPPIPDTFYCEICRLSRADPFSVSMMHPLHPVKLSTTLVPTEGSNPMQSVEKTFQLARAHKDIVLKSEFDIQAWCMLLNDKVPFRMQWPQYADLVVNGYSVRAINRPGSQLLGANGRDDGPIITPYIKEGVNKISLTGCDTRIFCLGVRIVRRRTLQQILNMIPKESDGERFEVALARVCCRVGGGNSADDAGSDSDLEVVSDTFSISLRCPMSGSRMKIAGRFKPCVHMGCFDLEVFVEMNQRSRKWQCPICLKNYALENIIIDPYFNRITSMMKNCGEEFTDVEVKPDGYWRVKAKSESECRELGNLAKWHCPDGSLPVSTSGEDKRVETLNVKQEGVSDSPNGLRLGIRKNCNGDWEVSKPKDTNISSDNRLNADLGNHEVVVIQMSSSGSESRLDGDDPSVNQSGGGHTDYSPTNGIETNSVCHTNVDSTYGYTIPNTSAPMANAEVIVLSDSEDDEILISPTVGYGNNQTGDAVDAYSVPPPGIMDPYAGDHSIGGNPCLGVFDNPNESIFGIPSVWPLHSGTQASSGFQLFSSDVDVSDALAHGDINCSSSLNSYTLAPDTALGSSALIPNSSTDRSDTDLNGGLVDNPLAFGGQDPSLQIFLPTRPAESSVQHELRNHTDVSNGVCTEDWISLSLGGGAGGSIGDASTTNGLNSRPQIQSREDAPDSLTDSLNEADLLLAETASLLRSVDDAESDKASRKRSDGPFSFPRQKRSVRPRLNLSIGSDSE

SEQ ID NO: 3 - protein, 875 aa, Oryza sativa
MADLVSSCKDKLAYFRIKELKDILNQLGLPKQGKKQDLIDRVLALLTDEQGQRHHGWGRKNSLTKEAVAKIVDDTYRKMQIQCAPDLATRSHSGSDFSFRPIEEAYDSFQPEAKVRCICSSTMVNDSMIQCEDQRCQVWQHLNCVLIPDKPGESAEVPPVFYCELCRLSRADPFWVTAGNPLLPVKFVSSGVTNDGTSVPQSVEKSFQLSRSDRETVQRQEYDLQVWCMLLNDKVQFRMQWPQYAELHVNGISVRVVTRPGSQLLGINGRDDGPLITTCSREGINKICLSRVDARTFCFGVRIAKRRTVAQVLNLVPKEAEGESFEHALARVRRCLGGGDTAENADSDSDLEVVAESVTVNLRCPNSGSRMRIAGRFKPCIHMGCFDLETFVELNQRSRKWQCPICLKNYSLESLMIDPYFNRITSLLRNCNEDVNEVDVKPDGSWRVKGDAASRELSQWHMPDGTLCNPKEDVKPAMQNGNEQMMEGTSDGQKSLKIGIKRNPNGIWEVSSKADDKKPSVVGNRMQNNSGFRALNNIMHMSNSPTSSYRDGEDPSVNQESNRHVDLSLNNGNNEFDSFSLNFGQACNTDDRPQQQHNATDVIVLSDSDEENDAMVCPPAVYDNTTTANGSGFPFTTNGIGYTERYQEDAGVGTSGLGLLSNNVDDFEMNNWQMHSSYQQPEQGFQFFGNDTDVHNTFVGSHNSFGLAPNDYSLDCNVGVEEASVTPALSVCRNSNEMHGSLVDNPLALVGDDPSLQIFLPSQPSSVPLQEELSERANAPNGVQSDDWISLTLAAGGGGNEEPAPADVNSQPQIPSTETGIEPLTDAASAFLSTNIERRSGADLNPRRIENIFSHPRQPRSVRPRLCLSIDTDSE

SEQ ID NO: 4 - protein, 813 aa, Oryza sativa
MALDPADDPLLADCKYKLNHFRIKELKDVLHQLGLPKQGRKQELVDKIIAVLSDQQEQDSRLNGLPNKKMVGKETVAKIVDDTFAKMNGSTNAVPASRNQTDSGHIVKPKRKSDDSAQLDVKVRCPCGYSMANDSMIKCEGPQCNTQQHVGCVIISEKPADSVPPELPPHFYCDMCRITRADPFWVTVNHPVLPVSITPCKVASDGSYAVQYFEKTFPLSRANWEMLQKDEYDLQVWCILFNDSVPFRMQWPLHSDIQINGIPIRVVNRQPTQQLGVNGRDDGPVLTAYVREGSNKIVLSRSDSRTFCLGVRIAKRRSVEQVLSLVPKEQDGENFDNALARVRRCVGGGTEADNADSDSDIEVVADSVSVNLRCPMTGSRIKIAGRFKPCVHMGCFDLEAFVELNQRSRKWQCPICLKNYSLDNIIIDPYFNRITALVQSCGDDVSEIDVKPDGSWRVKGGAELKGLAQWHLPDGTLCMPTDTRSKPNIRIVKQEIKEEPLSEETGGRLKLGIRRNNNGQWEINKRLDSNNGQNGYIEDENCVVSASNTDDENSKNGIYNPEPGQFDQLTSNIYDLDSSPMDAHFPPAPTEQDVIVLSDSDDDNVMVLSPGDVNFSSAHDNGNAFPPNPPEASGICGEQPRGAGPDVTSFLDGFDDLELPFWESSSSQDAAGTQVTDNQCEMQNFIVNHQFLHEPILGVNLGGTAASNTLECEHDGALQACQSSDQDGDQNQTCHDGHSGDLTNLSIISTQDSLTNGKNASQKRTNCEDGTAGLDGSVVRSANGLRGEMPPLGQEQDRTVRQKLILTIESDSD

SEQ ID NO: 5 - protein, 48 aa, Oryza sativa
MAPSVMASSATTVAPFQGLKSTAGMPVARRSGNSSFGNVSNGGRIRCM

SEQ ID NO: 6 - DNA, 28 bp, Artificial
  PCR primer
gagatctgagtagggaggcgggcgaacc

SEQ ID NO: 7 - DNA, 30 bp, Artificial
  PCR primer
atctccagacgaccgataaccccacctcag

SEQ ID NO: 8 - DNA, 25 bp, Artificial
  PCR primer
acttggatgatggcatatgcagcag

SEQ ID NO: 9 - DNA, 24 bp, Artificial
  PCR primer
gtctgcaccatcgtcaaccactac

SEQ ID NO: 10 - DNA, 21 bp, Artificial
  PCR primer
gtccagctgccagaaacccac

SEQ ID NO: 11 - DNA, 25 bp, Artificial
  PCR primer
gtgaagatcagcgatgccaagtgtg

SEQ ID NO: 12 - DNA, 24 bp, Artificial
  PCR primer
ctctgcagtgtctccacctccgag

SEQ ID NO: 13 - DNA, 13128 bp, Artificial
  pHL080 sequence
taaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagcttctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatcccaccgcggtggcggccgctctagaaggatcgatctgagtagggaggcgggcgaaccgaggcggcggcgccgatggcggacctggtttccagctgcaaggataaactggcatactttagaataaaggaactcaaagatatattaaatcagctcggcttaccaaagcaaggaaagaagcaggatcttattgatagggtgttggcactcttaacagatgagcaaggtcaaaggcatcatggatggggaaggaaaaattctctcaccaaggaggcagtggcaaaaattgttgatgatacatacaggaaaatgcaaatccaatgtgctcctgatctagccaccaggagccacagcggatcagatttcagtttcaggcctatagaggaagcctatgactctttccagccagaggccaaagttcgctgcatttgcagtagcacaatggttaatgacagcatgatccagtgtgaagatcagcgatgccaagtgtggcaacatttgaattgtgtactcattccagataagcctggggagagcgctgaagttccacccgttttctactgtgaattatgccgactgagtcgggcagacccattttgggtcactgctggaaatccattactcccagtgaaattcgtgtcatctggtgttacaaatgatggaacaagtgtacctcaaagtgtggagaaaagcttccagctttctcgatcagatagagaaactgtccagagacaagaatatgacctccaggtttggtgcatgcttttgaatgacaaagttcagttcaggatgcagtggccccaatatgcagaattgcatgttaatggtatttctgtacgagtagtgactagacctggttctcaattactagggataaatggacgggatgatggtccactgataaccacatgcagtagagagggaattaataaaatttgcttatcaagggtcgatgctcggacattttgctttggtgttcgaattgctaaacggaggactgttgctcaggttttgaacttagttccaaaggaagctgaaggagagtcttttgagcatgctcttgctcgtgttcggcgctgtctcggaggtggagacactgcagagaatgctgatagtgacagtgatttggaagtggttgcggagtctgttacagtcaaccttcgttgccctaatagtggatccagaatgaggattgctgggagattcaagccttgcattcacatgggttgttttgatcttgaaactttcgtggaattgaatcaacggtcccgcaagtggcaatgtccaatatgtttaaagaattactctcttgagagcttgatgattgatccttacttcaataggattacttctctgttgcgcaattgcaatgaggatgtcaatgaggttgatgttaagcctgacggatcttggcgtgtgaagggtgatgctgcaagtagagaattatctcagtggcatatgcctgatggtaccctttgtaatcctaaggaagatgtcaaacctgccatgcaaaatggaaatgaacaaatgatggaaggtacttctgatggacagaaatctttgaaaattggaataaagagaaatccaaatggaatctgggaagttagtagcaaagcagatgacaagaagccttctgtggttggaaatcgcatgcaaaacaatagtgggttccgagctctaaacaacattatgcatatgagcaatagcccaactagtagttatagagacggggaagacccaagtgtgaaccaagagagcaataggcatgttgacttatcattgaacaatggtaataatgagtttgacagtttctctctcaattttggccaagcatgcaatacagatgatagaccacagcaacaacataatgccacagacgtcattgttcttagtgattctgatgaagagaatgatgctatggtttgtccaccagctgtctatgacaatactaccactgcaaatggtagtggttttcctttcaccactaatggtattggatatactgaaaggtaccaggaagatgccggcgttggtacaagtggccttggtttattgagtaacaatgttgatgattttgagatgaataactggcaaatgcattcttcttatcaacaacctgaacaaggcttccagttttttgggaatgatactgatgtccataatacttttgttggttcacacaattcctttggcttagcaccaaatgactattctcttgattgtaatgttggcgtagaggaggcttcggtaactcctgctctttcagtctgccggaatagtaatgaaatgcatggaagtttggttgataacccactggctttggttggcgatgatccatccttgcaaatttttcttccaagtcaaccttcctctgttcctcttcaggaagaacttagcgagcgtgctaatgcaccaaatggggttcagtctgatgattggatatcccttacactcgcagcgggtggaggtggtaacgaagagcctgcacctgctgatgtcaattcacagccacaaattccatcaacagagacagggatcgaaccattgaccgatgctgcttctgcatttctgagcacaaacattgaaagacgtagcggagctgatttaaatccaagaaggatagaaaatatattttctcatcctcgccagcctcggtctgttaggcctcgactctgtttatcaatagatactgattctgagtagtttggacatcataacggggtaactgagtttgcattagtttggcaaagctgccatccagaaatcatgatttatactgaggtggggttatcggtcgtctggagatccagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagaagcttgcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagaggatcgatccccggggatctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgatgacccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattagcttgcatgcctcgagtctagaggatccccggggtaccgagctcgaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactagtggttcccctcagcttgcgactagatgttgaggcctaacattttattagagagcaggctagttgcttagatacatgatcttcaggccgttatctgtcagggcaagcgaaaattggccatttatgacgaccaatgccccgcagaagctcccatctttgccgccatagacgccgcgccccccttttggggtgtagaacatccttttgccagatgtggaaaagaagttcgttgtcccattgttggcaatgacgtagtagccggcgaaagtgcgagacccatttgcgctatatataagcctacgatttccgttgcgactattgtcgtaattggatgaactattatcgtagttgctctcagagttgtcgtaatttgatggactattgtcgtaattgcttatggagttgtcgtagttgcttggagaaatgtcgtagttggatggggagtagtcatagggaagacgagcttcatccactaaaacaattggcaggtcagcaagtgcctgccccgatgccatcgcaagtacgaggcttagaaccaccttcaacagatcgcgcatagtcttccccagctctctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgagaaccagtaccagtacatcgctgtttcgttcgagacttgaggtctagttttatacgtgaacaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaatcttcatatgacgcctaacgcctggcacagcggatcgcaaacctggcgcggcttttggcacaaaaggcgtgacaggtttgcgaatccgttgctgccacttgttaacccttttgccagatttggtaactataatttatgttagaggcgaagtcttgggtaaaaactggcctaaaattgctggggatttcaggaaagtaaacatcaccttccggctcgatgtctattgtagatatatgtagtgtatctacttgatcgggggatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggggggggggggggggggttccattgttcattccacggacaaaaacagagaaaggaaacgacagaggccaaaaagctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtccccccccccccccccctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaa

SEQ ID NO: 14 - DNA, 2809 bp, Artificial
  cDNA clone sequence of OsSIZ1 gene
tggcggccgcgggaattcgatgagatctgagtagggaggcgggcgaaccgaggcggcggcgccgatggcggacctggtttccagctgcaaggataaactggcatactttagaataaaggaactcaaagatatattaaatcagctcggcttaccaaagcaaggaaagaagcaggatcttattgatagggtgttggcactcttaacagatgagcaaggtcaaaggcatcatggatggggaaggaaaaattctctcaccaaggaggcagtggcaaaaattgttgatgatacatacaggaaaatgcaaatccaatgtgctcctgatctagccaccaggagccacagcggatcagatttcagtttcaggcctatagaggaagcctatgactctttccagccagaggccaaagttcgctgcatttgcagtagcacaatggttaatgacagcatgatccagtgtgaagatcagcgatgccaagtgtggcaacatttgaattgtgtactcattccagataagcctggggagagcgctgaagttccacccgttttctactgtgaattatgccgactgagtcgggcagacccattttgggtcactgctggaaatccattactcccagtgaaattcgtgtcatctggtgttacaaatgatggaacaagtgtacctcaaagtgtggagaaaagcttccagctttctcgatcagatagagaaactgtccagagacaagaatatgacctccaggtttggtgcatgcttttgaatgacaaagttcagttcaggatgcagtggccccaatatgcagaattgcatgttaatggtatttctgtacgagtagtgactagacctggttctcaattactagggataaatggacgggatgatggtccactgataaccacatgcagtagagagggaattaataaaatttgcttatcaagggtcgatgctcggacattttgctttggtgttcgaattgctaaacggaggactgttgctcaggttttgaacttagttccaaaggaagctgaaggagagtcttttgagcatgctcttgctcgtgttcggcgctgtctcggaggtggagacactgcagagaatgctgatagtgacagtgatttggaagtggttgcggagtctgttacagtcaaccttcgttgccctaatagtggatccagaatgaggattgctgggagattcaagccttgcattcacatgggttgttttgatcttgaaactttcgtggaattgaatcaacggtcccgcaagtggcaatgtccaatatgtttaaagaattactctcttgagagcttgatgattgatccttacttcaataggattacttctctgttgcgcaattgcaatgaggatgtcaatgaggttgatgttaagcctgacggatcttggcgtgtgaagggtgatgctgcaagtagagaattatctcagtggcatatgcctgatggtaccctttgtaatcctaaggaagatgtcaaacctgccatgcaaaatggaaatgaacaaatgatggaaggtacttctgatggacagaaatctttgaaaattggaataaagagaaatccaaatggaatctgggaagttagtagcaaagcagatgacaagaagccttctgtggttggaaatcgcatgcaaaacaatagtgggttccgagctctaaacaacattatgcatatgagcaatagcccaactagtagttatagagacggggaagacccaagtgtgaaccaagagagcaataggcatgttgacttatcattgaacaatggtaataatgagtttgacagtttctctctcaattttggccaagcatgcaatacagatgatagaccacagcaacaacataatgccacagacgtcattgttcttagtgattctgatgaagagaatgatgctatggtttgtccaccagctgtctatgacaatactaccactgcaaatggtagtggttttcctttcaccactaatggtattggatatactgaaaggtaccaggaagatgccggcgttggtacaagtggccttggtttattgagtaacaatgttgatgattttgagatgaataactggcaaatgcattcttcttatcaacaacctgaacaaggcttccagttttttgggaatgatactgatgtccataatacttttgttggttcacacaattcctttggcttagcaccaaatgactattctcttgattgtaatgttggcgtagaggaggcttcggtaactcctgctctttcagtctgccggaatagtaatgaaatgcatggaagtttggttgataacccactggctttggttggcgatgatccatccttgcaaatttttcttccaagtcaaccttcctctgttcctcttcaggaagaacttagcgagcgtgctaatgcaccaaatggggttcagtctgatgattggatatcccttacactcgcagcgggtggaggtggtaacgaagagcctgcacctgctgatgtcaattcacagccacaaattccatcaacagagacagggatcgaaccattgaccgatgctgcttctgcatttctgagcacaaacattgaaagacgtagcggagctgatttaaatccaagaaggatagaaaatatattttctcatcctcgccagcctcggtctgttaggcctcgactctgtttatcaatagatactgattctgagtagtttggacatcataacggggtaactgagtttgcattagtttggcaaagctgccatccagaaatcatgatttatactgaggtggggttatcggtcgtctggagatctcatcactagtga

SEQ ID NO: 15 - DNA, 3371 bp, Oryza sativa
aaaccacaacgaactaccccctcctcgtcgagccgacgcgagagaggaaagtgggttgcggcttgctgcgcgtgtggagtcgccattccccaattcgctgcgccgcccgccggatctcgtcttgccccctgcggcggcggtttgggcccccccttccgatcggtttcccccccgcacatggtcgtggcggcggcggaggtggtggtggtgcgggagtagggaggcgggcgaaccgaggcggcggcgccgatggcggacctggtttccagctgcaaggataaactggcatactttagaataaaggaactcaaagatatattaaatcagctcggcttaccaaagcaaggaaagaagcaggatcttattgatagggtgttggcactcttaacagatgagcaaggtcaaaggcatcatggatggggaaggaaaaattctctcaccaaggaggcagtggcaaaaattgttgatgatacatacaggaaaatgcaaatccaatgtgctcctgatctagccaccaggagccacagcggatcagatttcagtttcaggcctatagaggaagcctatgactctttccagccagaggccaaagttcgctgcatttgcagtagcacaatggttaatgacagcatgatccagtgtgaagatcagcgatgccaagtgtggcaacatttgaattgtgtactcattccagataagcctggggagagcgctgaagttccacccgttttctactgtgaattatgccgactgagtcgggcagacccattttgggtcactgctggaaatccattactcccagtgaaattcgtgtcatctggtgttacaaatgatggaacaagtgtacctcaaagtgtggagaaaagcttccagctttctcgatcagatagagaaactgtccagagacaagaatatgacctccaggtttggtgcatgcttttgaatgacaaagttcagttcaggatgcagtggccccaatatgcagaattgcatgttaatggtatttctgtacgagtagtgactagacctggttctcaattactagggataaatggacgggatgatggtccactgataaccacatgcagtagagagggaattaataaaatttgcttatcaagggtcgatgctcggacattttgctttggtgttcgaattgctaaacggaggactgttgctcaggttttgaacttagttccaaaggaagctgaaggagagtcttttgagcatgctcttgctcgtgttcggcgctgtctcggaggtggagacactgcagagaatgctgatagtgacagtgatttggaagtggttgcggagtctgttacagtcaaccttcgttgccctaatagtggatccagaatgaggattgctgggagattcaagccttgcattcacatgggttgttttgatcttgaaactttcgtggaattgaatcaacggtcccgcaagtggcaatgtccaatatgtttaaagaattactctcttgagagcttgatgattgatccttacttcaataggattacttctctgttgcgcaattgcaatgaggatgtcaatgaggttgatgttaagcctgacggatcttggcgtgtgaagggtgatgctgcaagtagagaattatctcagtggcatatgcctgatggtaccctttgtaatcctaaggaagatgtcaaacctgccatgcaaaatggaaatgaacaaatgatggaaggtacttctgatggacagaaatctttgaaaattggaataaagagaaatccaaatggaatctgggaagttagtagcaaagcagatgacaagaagccttctgtggttggaaatcgcatgcaaaacaatagtgggttccgagctctaaacaacattatgcatatgagcaatagcccaactagtagttatagagacggggaagacccaagtgtgaaccaagagagcaataggcatgttgacttatcattgaacaatggtaataatgagtttgacagtttctctctcaattttggccaagcatgcaatacagatgatagaccacagcaacaacataatgccacagacgtcattgttcttagtgattctgatgaagagaatgatgctatggtttgtccaccagctgtctatgacaatactaccactgcaaatggtagtggttttcctttcaccactaatggtattggatatactgaaaggtaccaggaagatgccggcgttggtacaagtggccttggtttattgagtaacaatgttgatgattttgagatgaataactggcaaatgcattcttcttatcaacaacctgaacaaggcttccagttttttgggaatgatactgatgtccataatacttttgttggttcacacaattcctttggcttagcaccaaatgactattctcttgattgtaatgttggcgtagaggaggcttcggtaactcctgctctttcagtctgccggaatagtaatgaaatgcatggaagtttggttgataacccactggctttggttggcgatgatccatccttgcaaatttttcttccaagtcaaccttcctctgttcctcttcaggaagaacttagcgagcgtgctaatgcaccaaatggggttcagtctgatgattggatatcccttacactcgcagcgggtggaggtggtaacgaagagcctgcacctgctgatgtcaattcacagccacaaattccatcaacagagacagggatcgaaccattgaccgatgctgcttctgcatttctgagcacaaacattgaaagacgtagcggagctgatttaaatccaagaaggatagaaaatatattttctcatcctcgccagcctcggtctgttaggcctcgactctgtttatcaatagatactgattctgagtagtttggacatcataacggggtaactgagtttgcattagtttggcaaagctgccatccagaaatcatgatttatactgaggtggggttatcggtcgtctggttgatgtaaagaaaaacaccagcattgatgctttgttgcctcaatggtttacaagctttcaagtgttctatttgatccaggagggatctgcatagaggacatctgatggttggtatagaaaattttctaactgggtatcgaggcttaatgtggcgactggagcagtttgtacattttttttgttttgacttttactggatataaggaatagaggtggggtcatccctggaccctggatgcaagacaaatacatgtgaatgttttgggtgtaccatcattgtatttagggtttgggggtactaatttagttgtttattaacctggtatttgatggagaataaattattcctggaagtggcggccaataaacagagtcgttggggttttacttgggt

SEQ ID NO: 16 - DNA, 3157 bp, Oryza sativa
actccagccgcccccctggctggctctgcctctggctccggttcttgccgctcgcgatccccaaagcgcggcaatccgccgcacgcgcgcgcgcgcgctcgcactccctccgccccgccgcgccgtccctcctgacgcggcgtctcgccgccgccgccgccgccgccaggttggtggtcgtcgcttcaggttcgcggaagccagcgcgtgggcaagcgtcctggcctccagggacgagccagggttcgccgggaaggaggacggtgagttcattccccatcggattttccagccatggcgctcgaccccgccgacgatccgctgctcgccgattgcaagtacaagttgaatcactttagaataaaagagctaaaggatgtcctgcatcagcttggacttccaaagcaaggaaggaaacaggaacttgtggacaaaattatagcagtattgtctgatcaacaagaacaagactccagattgaatggcttgccaaacaaaaagatggttgggaaagaaactgtggctaaaatagttgatgacacttttgcaaaaatgaatggctccacaaatgctgttccagcctctagaaatcagactgactcggggcacattgtaaagcctaaaaggaagtcagatgattctgctcagttggatgtcaaagtccgttgtccctgtggttattccatggccaatgattccatgattaagtgtgaaggtccacaatgcaatacacagcaacatgtaggctgcgttatcatatctgagaagcctgcagacagtgttcctccagaattaccaccacacttttattgtgatatgtgccgaatcactcgagctgaccctttctgggttactgttaatcatcctgtacttccagtctcaataactccctgtaaagtagcatctgatgggtcatatgctgtacagtatttcgagaagacctttccgctatcaagagctaattgggagatgcttcagaaagatgaatatgacctccaggtttggtgtatcctattcaatgatagcgtacctttcaggatgcagtggcctttacattctgatattcaaattaatggtattcctattagggttgtcaacaggcaacccacacaacagttaggggtgaatggtagagacgatggtccagttttaacagcatatgtgagagaagggtctaataagattgtcctatctagaagtgactctcgcacattctgtttgggagtcaggattgccaagaggagatctgtagaacaggtcctatctttggtgccaaaggaacaagatggtgagaactttgataatgcccttgctcgtgtgcgtcgctgtgttggtggtggaaccgaggcagataatgctgacagtgacagtgatattgaagttgtagctgattctgtctctgtgaatctccgatgccctatgactggttcaaggatcaagatagctggccggttcaaaccctgcgttcacatgggttgttttgatttagaagcttttgtggaacttaatcaacgttcaagaaagtggcagtgcccaatttgcctgaaaaactactctttggacaacataatcattgatccttacttcaaccgcataactgctttggtccaaagttgtggagatgatgtatctgaaattgatgtcaagcctgacggttcctggagagtcaagggtggagcagaactgaagggtcttgcacagtggcatttacctgatggcactctctgtatgcctacagatacaaggtccaagcccaacataagaattgtaaagcaagagattaaagaagaaccgttgtctgaagaaacaggtggccgtcttaagttgggaatcagaagaaacaacaatggccagtgggaaattaacaagagattggattctaacaatggtcagaatggatatattgaagatgaaaactgtgttgtttcagcaagcaacactgatgatgagaacagcaaaaatggaatctataatccagaaccagggcaattcgatcaactaaccagcaatatctatgatcttgattcttctcctatggatgcacattttcctccagcaccaacagagcaggatgtaatagttctgagtgactcagatgatgataatgttatggtgttatcacctggtgatgttaatttcagttcagcacatgacaatgggaatgcattcccacccaacccacctgaagcttcaggaatttgtggggaacaacctagaggagccggcccagatgtgacttcatttcttgacggtttcgatgatctggaactgccattttgggaatcttcgagctctcaagatgctgcaggcacacaggtgacagacaatcaatgcgaaatgcaaaatttcattgtcaaccatcaatttttgcacgagccaattttaggggttaacttgggtggaacagcagcatcaaacacactggaatgtgagcatgatggtgcgttacaagcttgtcagtcgagtgatcaagatggtgatcagaatcaaacatgccatgatgggcactccggggatttgacaaatcttagtatcattagcactcaagatagtttaactaatggcaaaaatgcttctcagaaaaggacaaactgtgaagatggcacggcaggtttagacggctcagttgtcaggagcgcgaatggcttgagaggagagatgccaccacttgggcaggagcaggatcgtacagttagacaaaagttgatattaacaatcgagtcagactctgattagcagctgatgctatttgatgtatggtccctgctatagtgctatgccccccagatgcatgcaccatcaccatcacttattttttggctgtgcactaagaagggcatcacttcttttttggttgtgccctagttgagaagggatagatgttggagctatggtggatggattgatgtttggcggcaagaagctgggtcggttatccttgcttctactaaggtggtattgattgatacagttaggtagggtaggtccattgtgtaggcagcaattttgtagctagcttgcatatacatcacaatggaattgcagtgacagaagttagctgatgtaaatatcatatgatgcttttctgttgcctggtgtacatattatatgatgttttttttttctgttggttggttacatatagagagttactccc

SEQ ID NO: 17 - protein, 681 aa, Sorghum bicolor
MSSGVGNDGASVPQIVEKTFQLSRADRETVQRPEYDLQVWCILINDKVQFRMQWPQYAELQVNGIPVRVMTRPGSQLLGINGRDDGPLVTTCSREGINKISLSRVDARTFCFGVRIVRRRTVPQVLNLIPKEGEGESFEDALARVRRCLGGGGATDNADSDSDLEVVTESVTVNLRCPNSGSRMRIAGRFKPCVHMGCFDLETFVELNQRSRKWQCPICLKNYSLENLMIDAYFNRITSLLQNCSEDVNELDVKPDGSWRVKGDAATRDLSQWHMPDGTLCDSKEDTNPGVTSVNEFKREGTSDGHRTLKIKKNPNGSWQVSSKADDKKPVVRHHIQNNNGFSTPNMPIISSPTGSYRDGEDASVNQEGGGIQFDIALNQEFDSFAHNFGQTYNTEDRQQPQHNAADVIVLSDSDEENDPIVRPPAVYANATTNGDSFPFVTDAAGTGYPERYQEDAGVGTSGLGLLNNNTGDFEINNWQMHSYPQPEQGFQFFGTDTDVGNPFVGPHNSFNIAPEDYSLDCNVGIEDPSAAHDVSICRNSNDVHGSLVDNPLALAGDDPSLQIFLPSQPSTVPLQEELSERANTPNGVHPDDWRISLTLAAGGGGNEESTSVDGLKSQPKVPSKEAGVEPLLDAASALPSMNRCNGSNLNPRRIENIFSHPRQPRSVRPRLCLSLDTDSE

SEQ ID NO: 18 - DNA, 2852 bp, Sorghum bicolor
cctcatttttattgtgaattgtgccgactgaaccgggcagacccgttttgggtgactacaggaaatccattactacctgtgaaatttatgtcatctggtgttggaaatgatggagcaagtgtacctcaaattgtggagaagaccttccagctttcccgagcagatagagaaacagtccagagacctgaatacgatctccaggtttggtgcattcttataaatgacaaagtccagttcaggatgcaatggcctcaatatgcagaattgcaagtgaatggtattcctgtacgagtaatgaccaggcctggttctcagttactagggataaatgggcgggatgatggaccactggtaaccacatgcagtagagaagggatcaataaaattagcttatcaagagttgatgcccgaaccttttgctttggagttcgaattgttaggaggaggactgttcctcaggtattaaacttgatcccaaaggaaggtgaaggggagtcttttgaggatgctcttgctcgtgtccgtcgctgtcttgggggtggaggtgctacagacaatgctgatagtgatagcgatctggaagtggttactgaatctgttacagtcaaccttcgttgccctaatagtggatccagaatgaggattgctggaaggttcaagccttgtgttcacatgggctgttttgatcttgaaacttttgtggaattgaatcaacgctcacgcaagtggcaatgcccaatatgtttaaagaattactctctcgagaacttgatgatcgatgcttatttcaatcggattacttctttgttgcaaaattgcagtgaagatgttaatgagcttgatgttaaacctgatgggtcttggcgtgtgaagggtgatgccgctaccagagatctatctcagtggcatatgcctgatggtactctttgtgactcaaaggaagatacaaaccctggtgtcacaagtgttaatgagttcaagagagagggtacttctgatggacatagaactttgaaaattaaaaaaaaccctaatggatcatggcaggttagcagtaaagcagatgataaaaaacctgtggttagacatcacatccaaaacaacaatgggttctcaacaccaaacatgcctattatcagtagccccactgggagttatcgagatggcgaagatgcaagtgtgaaccaagaagggggtggtattcaatttgatatagcattgaaccaagagtttgacagttttgcacataactttggtcagacatacaatacagaggatagacaacagccacaacataatgctgcagatgtcattgttcttagtgattctgatgaagaaaatgacccgattgttcgcccgccagctgtctatgcaaatgcaactacaaatggtgacagttttcctttcgtcactgatgctgctggaactggatatcctgaaaggtaccaggaggatgctggcgttggtacaagtggccttggtttattgaacaacaatactggtgattttgaaataaataactggcaaatgcattcttatccacaaccggagcaagggttccagttttttgggactgatactgatgttggcaatccttttgttggtccacataattccttcaatattgcaccagaagactactcgcttgactgtaatgttggcatagaggatccctctgcagctcacgatgtttcaatttgccgaaacagtaatgatgtgcatggaagcttggttgataacccattggctttagcaggcgacgatccatctttgcaaatttttcttccaagtcaaccttccactgttccccttcaggaagaattgagtgagcgtgctaatactccaaatggagtccaccctgacgattggaggatatctcttacgcttgcggccggtggagggggtaatgaagagtctacaagtgttgatggtctaaaatcacagccaaaagttccatcgaaagaggcaggagtcgaacctttgcttgatgctgcttctgctctcccaagcatgaacagatgtaatggatctaatctaaatccaagaaggattgaaaatatattttctcaccctcgccaaccgcggtctgttaggcctcgtctgtgtttgtcattagatactgattcagagtagtttggacatcacactgggatagctgaatttgctactaatctggcaaagttggcttccagaatcttgatttttactgtggagggctcaggttattgttgttgatacaaagagaaacaccagtgtggatgctttgttgcattaacctgggaagattcaagtgttctatatgatccaagtgggatctgcatagagttcatctaatggttggtacaggatattttctaactgggagtggtttaatgtggctagtggagcaggctgtacattttccctgacatttactgggtataaggaatagggggccatcacttggcataagacaaatacatgtgaatgttttgtgtgtaccatcattgtatttagggtttgggtgctaatttagatgtttattattaccctgatgtttgatggaaaataaattattccttgaagtggcggtcaataaacagagcccccttcggttttgagtcggagctgctgaccgttcatctgttcttgttgttgttgtgaagtggtagcttcgttcagcttcagcttccatggtgggaccaatcatgccaaagttgtgtctagcagagggcaatctccatggtgtcttgcaatacaagtactcgtatattttgctctccaggatgataagtttctttgtgcatcatgctgtattatgccggtgttccccttctcatttcttgcttcactgaactgcttggcatagtttg

SEQ ID NO: 19 - protein, 876 aa, Vitis vinifera
MDLVTSCKDKLAYFRIKELKDVLTQLGLSKQGKKQDLVDRILAILSDEQVSRMWAKKNAVGKEEVAKLVEDTYRKMQVSGATDLASKGQVLSDSSNVKFKEELEDSYNDMKIRCPCGSALPNETMLKCDDLKCQVWQHIGCVIIPEKTMEGIPPTPDPFYCEICRLSRADPFWVTVAHPLLPVKLTTTSIPTDGTNPVQSVEKTFHLTRADRDMVSKHEYDVQAWCILLNDKVSFRMQWPQYADLQVNGMAVRAINRPGSQLLGANGRDDGPVITPCTKDGINKISLTGCDARIFCLGVRIVKRRTVQQILSLIPKESDGERFEDALARVRRCIGGGGATDNADSDSDLEVVADFFTVNLRCPMSGSRMKVAGRFKPCAHMGCFDLEIFVEMNQRSRKWQCPICLKNYSLENVIIDPYFNRITSSMQSCGEDVTEIQVKPDGCWRVKPENERGILAQWHNADGTLCPLAEGEFKPKMDVLKQIKQEGISECHSSLKLQIKNRNGVWEVSKPDEMNTLTCNRLQEKFEDPGQQVIPMSSSATGSGRDGEDPSVNQDGGGNYDFSTNPGIELDSISLNIDNNAYAFPERNTPAPMGDTELIVLSDSEEENDTLMSSGTLYNNSRADAGGINFSIPTGIPDSYAEDPTAGPGGSSCLGLFSTADDDFGMSGSLWPLPPGTQPGPGFQFFGTDTDVSDALADLQHNPINCPTSMNGYTLGPEVVMGSAALVPDPSIGRTDTDMNDGLVDNPLAFGGDDPSLQIFLPTRPSDASVPTDLRNQADVSNGSRPDDWISLRLGGSSGGHAESPAANGLNTRQQLPSKDGDMDSLADTASLLLGMNDGRSDKTSSRQRSDSPFSFPRQRRSVRPRLYLSIDSDSE

SEQ ID NO: 20 - DNA, 3162 bp, Vitis vinifera
atggatttagtgacttcatgcaaggacaaattggcatattttcgaataaaggagctcaaggacgtactgactcaacttggtctttcaaagcagggaaagaagcaggatctcgttgatcggatattagccattctctctgatgaacaagtttccaggatgtgggcaaagaagaatgctgttgggaaggaagaagtagcaaaactagttgaggatacttacagaaaaatgcaggtatctggagccactgacttagcatcaaagggacaggttctctcagatagcagtaatgtcaaattcaaagaagaacttgaggattcatataatgatatgaagattcgttgtccatgtggaagcgcactgccaaatgagacaatgcttaagtgcgacgatctaaaatgccaggtgtggcagcatataggttgtgttataattccagagaaaactatggagggtattccaccaactcccgacccattctactgtgaaatttgtcgactaagtcgagctgaccctttttgggttactgtggcacatcctttacttcctgtgaagttgacaacaactagtattccaactgatggtacaaacccagtgcagagtgttgagaaaacatttcatctcacaagggctgacagagacatggtatcaaaacatgagtatgatgttcaggcttggtgtattctccttaatgacaaggtttcatttaggatgcagtggccacaatatgcagacctacaagtcaatggtatggcagttcgtgctatcaatagacctggctcacagttgctaggtgctaatgggcgtgatgacggacctgttatcacaccatgtaccaaagatggaattaataagatttccttaacgggatgcgatgctcgtattttctgtttaggggttagaattgtaaagcggcgaactgttcaacagattttaagcttgattcctaaagaatcagatggtgagcgttttgaagatgcgctggctcgtgttcgtcgttgcattggtggtggaggtgcaactgataatgctgatagtgacagtgacctggaagtggttgcagatttctttactgtcaatctacgatgtcctatgagtggttcaagaatgaaggttgctggaagattcaaaccttgtgctcacatgggctgttttgatcttgaaatttttgtggaaatgaaccaacgttctaggaagtggcaatgtcccatttgtctcaagaactattctctagagaatgttatcattgatccatatttcaatcgcatcacatcctcgatgcagtcttgtggagaagatgtaactgagatacaagtgaagcctgatggttgttggcgtgtaaagcctgagaatgaacgtgggattctagcacagtggcacaatgctgatggtactctctgtccccttgctgagggagaatttaaaccaaaaatggatgtgttgaagcaaatcaaacaggaaggaatttcagaatgtcattccagtttgaaactccaaattaagaatcgcaatggggtttgggaagttagcaaacctgatgaaatgaataccctcacttgtaatagactacaagaaaagtttgaagaccctggtcagcaagttatcccaatgagcagcagtgccactggaagtggtagggatggtgaggatccgagtgtaaatcaggatggtggtgggaattatgatttttccaccaaccccgggattgagcttgattccatttctctaaacatagacaacaatgcctatgcatttccagagcgaaatactcctgcaccaatgggggatacagagcttattgttctcagtgattcagaagaagagaatgacactttgatgtcttctggaaccctttacaacaacagtagagctgatgctggtgggattaatttctcgattcctactggtattccggattcatatgcagaagatcccactgctgggcctggtgggagttcatgcttgggtctttttagtactgccgatgatgattttgggatgtctggatccctctggccattacctcctggtactcaaccaggccctggtttccaattttttggtacagatacagatgtctcagatgctttagctgatttgcagcataatcccatcaactgtcccacatcaatgaatggctacacattgggtccggaggttgtcatgggatctgctgctctagttcctgatccttccattggtcgtactgataccgacatgaatgatggcttggttgataatccgttggcctttggtggggatgacccatctcttcaaatatttcttcctacaaggccctccgatgcatcagtgcctaccgatttgagaaatcaagctgatgtgtcaaatggtagccgacctgatgattggatttctctgaggcttgggggtagcagtgggggtcatgctgaatctccagctgcaaatggattgaataccagacagcaattaccatccaaagatggtgacatggattctttggctgacactgcttctttgcttcttggtatgaatgatggtagatctgacaagacaagtagcaggcaacgatcagatagccctttttcgttccctcgccagcgacgctctgtgaggccaaggctgtatctttcgattgactcagattctgagtagagttgtttgctgagcattaccgtagccctcttgaaaattattggagatgctaccggatataacccctccccttgtgaagaggacatttcctggagtgatatgttgacccatctcttattatcaactcggtttatatttagaagaaagctttgaaggttttcatcatcaagaagtgccaagcctgaatacgggtgctaggcatgcacaggacactaagaaacgtggacgcttttaaagaacaaatgtgccggcacctgcggaatttcttgctattgggctgctacaaattcttttgccctttttgtcaatgaagatctgcaggtccagtgctggaaaattgtacattagtcatttgactggaaaccgaaaagtgaatccattgggtaaacctttgggtgtagaagggtagtccctttcagtaacacaagatataatgccctttttgtctgttttatcatcacagcacattagattgtaaaaatgccttcaccatctcagattcagatatttatagagaaacatttatttttc

SEQ ID NO: 21 - DNA, 3219 bp, Arabidopsis thaliana
gtctgggtttggttgtctttttattttcctcttccttggccaaagaaaatcttatcgtagctgtgaaaaccctagtttctaatcatctcaattctctcattaacctatcaatttcgatagggtttgatcaagtatccgatttcatgatcggttgctgtatggagactaattgctggagtttaggttcgtgtgttttgaactgagcttttggttcttgtttgtgtctggtgttgaagacatggatttggaagctaattgtaaggaaaaactttcatattttcggataaaagagctcaaggatgtgctcactcagctgggactttcgaaacagggaaagaagcaggaacttgtcgaccggatcttgacccttctttctgatgaacaagctgccaggttgttgtctaaaaagaatacagtggcaaaggaagcagttgccaaattagtggacgatacatataggaaaatgcaagtatctggggcaagtgatttagcatcaaaaggacaagtgagttcagataccagtaatctgaaagttaagggagagcctgaagaccccttccaaccagaaattaaagttcgatgtgtttgtggaaactcgctagaaacagactcaatgatacagtgtgaggatccaagatgccatgtttggcagcatgttggctgtgttattctcccagataagcctatggatgggaatccaccacttccggaatcattttattgtgaaatctgccgacttactcgagctgacccattttgggttacagtggcacatccactctctccagtgaggctgactgcaacgactatcccaaatgatggtgcaagcacaatgcagagtgtggagagaacatttcaaatcacaagggcagacaaggaccttttggccaaaccagagtacgatgttcaggcttggtgtatgctcttgaatgataaagttctctttaggatgcagtggcctcagtatgctgatctgcaggtcaatggtgtgcctgtacgtgcaattaatcgacctggaggacagcttttgggagtcaacggccgcgacgatggacccattattacatcttgtattagggatggagttaacagaatatccttgagtggaggtgacgttcggattttttgttttggggtcagacttgtgaagcgcaggactctacaacaggttctaaatttgattccagaagagggtaaaggggagacttttgaagatgctcttgcacgtgtccgccgatgcattggaggtggaggtggagatgataatgccgacagtgatagtgacattgaagttgttgctgatttcttcggtgtcaatcttcggtgtcctatgagcggttctaggataaaagttgctgggagatttttaccctgtgtgcacatgggctgttttgaccttgatgtgtttgtggagttgaatcaacgttccagaaagtggcagtgccctatttgtctgaagaactactcagtggagcatgtaatcgtcgatccttattttaaccgtatcacgtctaagatgaagcattgtgatgaagaggtgactgaaattgaagtgaaacctgatggttcttggcgtgtaaagttcaaaagagagagtgagcgaagggaactgggggaactctcacagtggcatgcacctgatggtagcctttgcccctctgctgttgatattaaacggaagatggaaatgttaccggttaagcaagaaggttactcagatggtccagccccgctaaaacttggaataaggaagaatcgtaatggcatttgggaagttagcaaacctaatacaaatggattatcttccagtaataggcaagaaaaggttgggtatcaggagaagaatattataccaatgagtagtagtgctactggaagtggtagggatggtgatgatgcaagcgtaaaccaggatgctattggaacttttgactttgtagccaacggcatggaacttgattccatttccatgaatgttgattcaggttataactttcctgacagaaaccaatctggcgagggtggaaataatgaagtcatcgttctgagtgattctgatgacgagaatgatttagtgatcactccagggcctgcatacagtggttgtcaaacagatggtggacttacttttccactgaaccctcctggaataattaactcatataatgaggacccacacagcatagctgggggaagttcaggcttaggtcttttcaatgatgatgatgaatttgatacgcccctttggtcatttccttctgaaactccagaagcccctgggttccaactatttagatctgatgctgacgtttcaggaggtttagttggtttgcatcatcatagtccactaaactgttctcctgaaataaatggaggttataccatggctcctgagacatcaatggcatctgttcctgtggttcctggctctactggccgatctgaagcaaacgatggcctagttgacaatcctcttgcatttggtagagacgatccctcacttcaaatatttttgccaacaaaaccagatgcttcagctcagtcgggttttaaaaaccaagctgatatgtcaaatggtctccgtagtgaagactggatctcgcttaggctaggcgatagcgcctctgggaatcatggagatcctgcaactacaaacgggattaactcaagccatcagatgtctacgagggaaggttctatggatactacaacagagactgcgtcgttgcttctgggtatgaatgacagtagacaagacaaggcaaagaagcaaagatcagataatccattttcatttcctcgccagaagcgttctgtaagacctcggatgtacctctccattgactcggattctgaaacaatgaacaggatcatcagacaagacaccggagtttaaacaagatttgcataattctctgtgcaggcaagaattgaaccggtattgatattttcacttgtatgatgttgttgactctctcttcaatatcggttcagaatcttggccttgtctgctacactgcaggatgtaatttgcaaagcgaagcactggctgatttagtttctctgatagaaaagaaaagtgggcacagtggttccgatttaattagtagtttgtatactcgaaataggttttttttgtgtggacgatgataaaattacttcgaagccaggagctatagagagatatagcaatgtaaattatgggctccaaattttatt

SEQ ID NO: 22 - protein, 876 aa, Ricinus communis
MDLVTSCKDKLAYFRIKELKDVLTQLGLSKQGKKQDLVDRILAVLTDEQVPKTSAKKSVVGKEEVAKLVDDIYRKMQVSGATDLASKGEGVLESSKPVIKGEIDDSFHFDTKVRCPCGSSLETESMIKCEDPRCRVWQHIGCVIIPEKPMEAIPQVPDLFYCEICRLCRADPFWVSVAHPLYPVKLTTNIQADGSTPVQSAEKTFHLTRADKDLLAKQEYDVQAWCMLLNDKVPFRMQWPQYADLQVNGVPVRAINRPGSQLLGINGRDDGPIITPCTKDGINKISLNGCDARIFCLGVRIVKRRTVQQILNMIPKESDGERFEDALARVCRCVGGGAADNADSDSDLEVVADSFAVNLRCPMSGSRMKVAGRFKPCAHMGCFDLEVFLEMNQRSRKWQCPVCLKNYSLENVIIDPYFNRVTSKMQHCGEDITEIEVKPDGSWRAKTKSEAERRDVGELAQWHNPDGSLCVPISGEHKSKVEMEKQIKQEGNSEGYNGTGLKLGIRKNRNGFWEVSKPEDVNTSSSGNRLPERFEIIEQKVIPMSSSATGSGRDGEDPSVNQDGGGNFDFTNNGIELDSLPLNVDSTYGFPDRNFSAPVEDPEVIVLSDSDDDNDILMTTGTVYKNSQTDDGGAGFSMPPNGISNPYPEDPTVGNGLGFLNPNDDEFGIPLWPLPPGSQAGPGFQLFNSDVPDALVDIQHGPISCPMTINGYTLAPETVMGPSSLVADSSVGRSDTDTNDGLVNNPLAFGGEDPSLQIFLPTRPSDASGQSDLRDQADVSNGVRTEDWISLRLGGGGATGSHGDSVSANGVNSRQQMPPRDGAMDSLADTASLLLGMNDGRSEKASRQRSDSPFQFPRQKRSIRPRLYLSIDSDSE

SEQ ID NO: 23 - DNA, 3988 bp, Ricinus communis
caccaaacaaaaaaagaagagagaaaagaagagaaagtcggcgttttagaaagaaactctgcaaaccctaaaacgattcctctcttcgattctttcattttgccgttatcgatcgctgtctcgagacagtgcgtggactggtttgtttggttaattgagtaatcgatgatttaaagtattgatttaggcactgaatttaaaagcttcaagttcttggagagtcaaattatggatttggtgactagttgcaaggacaaattggcctatttccgtatcaaggagctcaaagatgtacttacacagctgggtctttcaaagcaggggaagaagcaggaccttgttgacagaatattagctgttctcacagatgaacaagtaccaaagacatcagcaaagaagagtgttgttggaaaggaagaggtggcaaaactagttgatgacatttacaggaaaatgcaggtttctggggccactgatctggcatctaagggggaaggtgttttggagagcagtaagccggtcattaaaggagaaattgatgattcctttcacttcgatacaaaagttcgctgcccatgtggaagctcattggagacagaatcgatgattaagtgtgaggatcctagatgtcgggtgtggcagcatataggttgtgttataattcctgaaaaacccatggaggctattccacaagttcctgacttgttttattgtgagatctgtcggctctgccgggctgaccctttctgggtttctgttgcacatcctctttatcctgtgaagttgactactaatattcaagctgatggctcaaccccagtgcaaagcgcggagaaaacatttcatcttactagggcagacaaggacttattggccaaacaagaatatgatgttcaggcttggtgtatgcttctgaatgataaggttccatttaggatgcaatggccacaatatgcagatttacaggtcaatggtgttcctgttcgtgccatcaataggcctggttcacaattattggggattaatggccgtgacgacggtccaattattacaccatgtacaaaagatgggattaataagatatcattaaatggatgtgatgcccgtatcttctgtttaggagttcgaattgtaaagcgacgaactgttcaacagatactcaacatgatacccaaggagtctgatggtgaacgctttgaagatgcactggctcgggtatgtcgttgtgttggtggtggagcagcagacaatgctgacagtgacagtgacttggaagtagttgcagattcttttgctgttaatcttcgttgtcctatgagtggttcgagaatgaaggttgctggaagattcaaaccttgtgctcatatggggtgtttcgatcttgaagtttttctggagatgaaccagcgttctaggaagtggcagtgccctgtttgtctcaagaactactcgttggaaaatgtaataattgatccatattttaatcgtgttacatctaagatgcagcattgtggtgaagatataactgaaatagaggtgaagcctgatggttcttggcgtgcaaaaactaaaagtgaagctgaacgtagggatgttggtgaacttgcacagtggcacaaccctgatggttctctgtgtgtacctatcagtggtgaacataaatctaaagtggaaatggaaaagcagatcaaacaggaaggtaattcagaaggttataatggtactggtttaaaacttggaatcaggaagaaccgcaatggcttttgggaagttagcaaacctgaggatgtgaacacctcctcttctggtaatagattgccggaaagatttgaaatcatcgagcagaaagttatccctatgagcagtagtgccactggcagtggtcgcgatggtgaagatcctagtgtaaaccaggatggtggtgggaattttgacttcacaaacaatgggatagaacttgattctttgcctctgaatgtagattcaacatatggatttcctgatcggaacttttctgcaccagtagaggatccagaagttattgttcttagcgattcagatgatgataatgatatattgatgacaactggaactgtttacaagaatagtcaaactgatgatggaggggctggtttttcaatgccccctaatggaatttcaaatccctatcctgaagatcctacagttggaaatggtttgggctttctcaatcctaatgacgatgaattcgggatacccctgtggccgttgccacctggaagccaagctggccctgggttccagttatttaactcagatgttccagatgccttagtagatatacagcatggtcctatcagctgtcccatgacaattaatggttatacattagctccagagactgtcatgggaccttctagtttagttgcagattcttctgttggtaggtctgatactgatacaaatgatggcttggtcaataaccccttagcgtttggtggtgaagatccctctcttcaaatctttcttccaactagaccttcagatgcatctgggcagtctgatcttagggatcaggctgatgtgtcgaatggtgtccgtacagaggattggatatctcttagacttggtggtggtggtgccactggcagtcatggtgattctgtttctgcaaatggagtgaattcaagacagcagatgcctccgagagatggtgccatggactctctggctgacaccgcttctctgcttcttggtatgaatgatggtagatctgagaaggcaagtcggcaaagatctgatagccctttccaatttcctcgtcaaaaacgttctataagaccacggttgtatctctcgattgactcggactcggagtagagaagtggagccttgttttcaaagataactgttgcttttcttatgaaattctttttgggagacatttccccaatgagggtatgatgaagaaaatcatcattttgtctctaggacaatgaagtctctgatgaggcacaaggcatttacagtgggaaaatgccatcaggaaataatcatcttgaagtagtgttggcacatgaagattccgccattggtgagctgtaggcctcgtaatctcccttctcaattttgtgcggaataacctttgtttctgatgttctttgttctatcagggttattacaagcactctcaattttcagcaaagatcatgatgtccagtgctggtaaattgtatagtagataggcatttgaatctgaaaattggattaatccatagatttggctcaaatcgtcggatgtagtggggaagccccgagttagtaacacaagtatccatctttttggtctctgtttaatgatgagagcaaatagcttgtaaatgtccctcagattcagttatatttatagaaagaataagaaaatcttaattgtgcgactctgcaacttagtagtgtactttggtcctgttcaataaccagggctcaaatgttgtatatataatgtttgttctagaagccattggcatgcaggtggcagggcacttgctgctaggtgatcctgtgtcaccatgaaatccagttagaaagatgacatacagcactttgacgagctctaaatttgcttctattgcttgctgtccgggatgatcggtatgaccctcaactcctcgttcctcccgtgtttcccctttgtttccttctcggtcttaactcttgtgggcgtaattgcagtttagcaaaccagtgcttgattgaatcaggcatgaactgggtggatagtcatgtatacacatattctggatggaatcttaagatgtttaaccactctacttattacgatgttgatattttacaggatttaggtgaattatttatagttttggattgtaaattgtcctggtgttttgtatggcttgctttcttaccgcatgcttctagagggcaaagaaaaatcatatatgaactcaatatcttctgggattaattttccatatccataaccactctt

SEQ ID NO: 24 - DNA, 3286 bp, Medicago truncatula
gcacgaggcaatactctctgccgcgtgatttcaacaaaccctaatctccaaaccctaatcacctccgattcattcttattacgattggttacttctgcggcgtcaccatgtgttgattattcgcttcttgattgagtctgttgattcagtttttgctgaattcttcttaggttttgtggaaatggatttggtagccggtatcaaggaaaaattaacatatttccgtataaaagagctcaaggatgtgctgactcagttaggactttcaaaacagggaaagaagcaggatctcgtcgatcggatattatccattctctcagatgagcaagtttccaaaatatgggccaagaagaatgctgttgggaaagagcaggtggcaaaattggtggatgacacatataggaaaatgcagatatcgggagccactgatctagcatcaaagggtcaggttgtgtcagatagtagtaatgtgaaggttaaagctgaagttgaagattcctttcaaattcaaactactacaactacaaagattcgctgtctctgtggaagtacattggaaacaggggatttgatcaagtgtgatgatgccagatgccaagtgtggcaacacatcagctgtgttattattccagagaaacctatggaaggcatcccaccagttcctgataaattttattgtgaactatgtcgactcagccgtgcagacccgttttgggtttcagtatctcatcctttgttacctgtaaagttggccacaaccagtattccaactgatggtaccaacccagtgcagtgcgtggagagaacatttcaactcacaagagcagacaaggacatggtatcaaaacaagaatttgatgttgaggcttggtgtatgcttctgaacgacaaggttccattcaggattcaatggccacagtatacagaccttgcagttaatggtcttcctattcgaacaactactagacccggttcacagttgcttggagctaacggtcgtgatgatggtccaattatcacgccgcatacaaaagacggaattaataagatttccttaacagtatgtgatgctcgcattttctgtttaggtgttcgaattgttagaaggcgcagtttgcaacagatcctaaacttaattccaaaggagtctgacggtgagccttttgaagatgctcttgcacgcgtctgtcgttgtgttgggggtggaaatgcagctgacaatgctgatagcgacagtgatttggaagtggtttcagatactttcagtataagccttcgttgtccgatgagtggttcaagaatgaagattggcggaagattcaaaccttgcattcacatgggttgttttgatcttgatgtttttgtggaaatgaatcaacggtcaagaaagtggcaatgtcctatatgtctcaaaaactatgcattagagaatatcatcattgacccttatttcaatcgcatcacttctatgatgattaattgcggtgaagatgttacagaggttgaggtgaagcctgatggctcttggcgtgttaaggcaaagagtgaaagtgaacgtctggatttagggattcttggccaatggcatcttcctaatggatctctttgtacttctactgctggagatatcaagagagtagaaacactgaagcaagtaaaacaggaaggtttttcagatggacctgctggtttaaaacttggcattaggaggaatcgcaatgggaattgggaagtcagtaagccagagacaaccaacacctcttctggtcatatattaaaagaggtttttggaaatcctgaacaagttgttattccaatgagcagcagtggctccgaaagtggtcgggatggtgatgatcccagtgttaaccagggtggtggtgggcatattgattattctactaccaatggaattgagatggattctcagtctcgcaataatgttgatttagctcgtggatatactgtgcataacacatctgctcaggtgggtggggcagagataattgttcttagcgactctgaagaagacaatgacatattggtgtctcctccaattgcaaataacaaccaccaaaatgatactgcagatggttactccatgccacctcctggaattgttgacccatacgttgaagatcagaatcttggtggaagttcatgcttggggctttttcctaatgaagatgattttggaatatcttccctgtggtcattgccttctgcatctcaggctggtccaggattccaattgtttggttctgatgcagatgcctctgatgcattggttcatttgcagcatgtccctattaattgcacctcatcactgaacggttatgcattggctcccgaaactgctttgggatctggcagtctcttacaagattcctctgctggacggtcagatgctgacttaaacggtggtttggttgacaacccattggcatttgctggagatgatccctctcttcagatttttctccccacaagaccagctgagtcgtctatgcagaatgaattgagagatcaagcaaatgtctctaatggtgtttctaccgaagattggacatcccttactctcggaggtggtgctggcggtagtaatggcgatgcttccactcaaaatggattgaattctagacaccaagtcccatccagagacaatggcacaaatactttggctgattctgcttctttgcttcttggtatgaatgatgtaagatctgacagagcaagtagaccaaggtcaggtagtcctttcacatttcctcgccaaaaacgttctgtaaggccccgcttgtacctttctattgattcggaatcggaataaaggtgtcagaacttgtgtctcaagaactttaggaactcttagcaccagatattgtatttagaaattgaaagaagttgggaggcatttttggtcctcgtaatgtggcagtatgtgttgaggcatctctggtggaagatataaattggccctaacgcttgcacctgccccctttggcaaatgatattggccgcaaagattcttgtttctccatgcttgatattgaaattgacgggttgtggtcaatctatcattcctttatttttcaattatctaccgagggtatagtgctggtttactgtatagttggtcattcaatctggtagtttaaacttcctaatttggtggggaaagtttcccattaggaatattttatctgcatttttggctctgcgcctgagagcaaataacctgtaaatgtttagccttcggattcaacaagagaattcaaatagtttt

SEQ ID NO: 25 - DNA, 3295 bp, Medicago truncatula
atggatgatttggtttcaagttgtaaggaaaaattgcaatattttcgtgtaaaggatctcaaagatgtgcttactcagataggaatttcgaagcagggaaagaagcaggatcttatcgataggatattatctatcatctcagatgaacaagttgctaaagtacgggctaagaagaatgctgttgaaaaagaacaagtggtaaaattagtggaggacacatatagaaaactgcaggtatctggagccactgatatagcatcaaaggggcaggttgcttcagacagcagtaatgtgaaaattaaaggtgaagttgaggattccgttcaatcagctacaaaggttcgatgtctctgtggaagttcattggaaacagatctattgatcaagtgtgaagatagaaaatgccctgtgtcgcaacatctcaactgcgttattattccggatacacccactgaaggactcccgccaatcccagatacattttattgtgaaatatgtcgtctcagtcgtgcagacccgttctcggtttcaatgatgcaccctttacatccggtgaagttgtctacaacccttgtcccaactgaaggatccaaccccatgcagagtgttgagaaaacatttcaacttgcaagagcacacaaggacatagtattaaaatcagaatttgatattcaggcttggtgcatgcttctgaacgacaaagttccattcaggatgcaatggccacaatatgcagacctagttgtaaatggctattctgttcgtgcaattaatagacccggttcacaattgcttggggctaatggtcgcgatgatggcccaattatcacaccatatataaaagaaggagttaacaagatttctttgacggggtgtgacactcgcattttctgtttgggggtccgcattgttagaaggcgcactttgcaacagatcttgaacatgattccaaaggagtctgatggtgagcgttttgaagttgctcttgctcgtgtctgttgtcgtgttggcggtggaaattcagctgatgatgctggtagtgacagtgatctggaagtagtttcagatacttttagcatcagccttcgttgtccaatgagtggttcaagaatgaagattgcaggaagattcaaaccttgtgttcacatgggttgttttgatcttgaagtttttgtggaaatgaatcaacgctcaagaaagtggcaatgccccatatgtctcaaaaactatgcactggagaatatcattattgatccttatttcaatcgcatcacttctatgatgaaaaactgtggggaggagtttacagatgtggaggtgaagcctgatggttattggcgtgtcaaggctaagagtgaaagtgaatgccgtgagttggggaatcttgctaaatggcactgtcctgacggatcgctccctgtttctaccagtggagaagacaagagagtggaaactttgaatgtcaaacaggaaggtgtttcagacagtcctaatggcttaagacttggcattaggaaaaactgtaatggagattgggaagtcagcaaacccaaggacacaaacatctcttctgataataggttgaatgctgatttaggaaatcatgaagttgtagttattcaaatgagcagcagtggctctgaaagtagattggatggtgatgatccaagcgtaaatcagagtggtggtgggcatacagattattctcctactaatgggattgagacgaattctgtgtgtcacactaatgttgattcaacttatggatataccattcctaacacttctgctccgatggctaatgcagaagttattgttcttagcgattctgaagatgatgaaatattaatatctcctacagttggttacggaaataatcaaactggtgatgcagttgatgcttactcagtgcctccgcctggaattatggatccatatgctggagatcacagtattggtggaaatccatgcttgggagtttttgataatcccaatgaaagcatttttgggattccctcagtctggccactgcattctggaactcaggcaagctcgggattccaactattcagttctgatgtggatgtgtctgatgcattggcccatggtgatattaattgctcctcttcactgaatagttatacgttggctcctgacactgctctgggatctagtgctctaataccaaattcatccactgatcggtccgacactgatttaaatggtggtttggttgacaatccattggcatttggtggacaggatccctcacttcagatttttctccctacaagaccagcagaatcatctgtgcagcatgaattaagaaatcacacagatgtgtcgaatggtgtctgcacagaggattggatctctcttagccttgggggtggggctggtggcagtattggtgatgcttcaacaacaaatggtttgaactccagaccgcaaattcaatccagagaagatgcgccagattctttaacagattctttaaatgaggctgatttgttacttgctgagactgcttctttgcttcgtagtgtggatgatgctgaatctgacaaagcaagtaggaaaagatcagacggccctttctcatttccccgccaaaaacgttcagtaaggccccgcttgaatctttctattggttcagattcagagtagagggtattacacctctattagctttagacgcccttaccgctacatttgtacctagaaactgaagaaagttagagcagcatttttagtcctcaggaagtgaaaactgttgtcttctgaaacatctatggtgtagccgcgctacaaaattttcgccgcaaaatcctgttcctgtagggtgtcattaccctacaggaacagaaagatgttggctgcaattgtcctcagttatttatgcagatataatcagagttgtcctaatgacacatgatagtgctgcttttgtgtagatgggcattcagatttgatagttcaaattattatatctgcaattttgtctctgctgctgatagcaattaaccaagtttcagctaaacaaattgaataggagaatgaaaaatagttttattatttacaaccaggcagatgctgcttttcgcttttgtaatatatatggttgctaatattttgtaatatatttaggcactagatagagtggtctcctggattagcaaattttcatgaggatcaaatcaacatttcctccttgtattcagttttagctcaactgtgttcttattctttggaacacaggttgaacgagttgaattgttgtaaaatatagaagctt

(19) United States
(12) Patent Application Publication
(10) Pub. No.: US 2011/0197316 A1
(43) Pub. Date: Aug. 11, 2011

(54) METHODS AND COMPOSITIONS FOR TRANSGENIC PLANTS WITH ENHANCED ABIOTIC STRESS RESISTANCE AND BIOMASS PRODUCTION

(75) Inventors: Hong Luo, Clemson, SC (US); Zhigang Li, Clemson, SC (US); Qian Hu, Clemson, SC (US)

(73) Assignee: Clemson University

(21) Appl. No.: 13/023,168

(22) Filed: Feb. 8, 2011

Related U.S. Application Data
(60) Provisional application No. 61/302,345, filed on Feb. 8, 2010.

(51) Int. Cl. A01H 1/00; C12N 15/82; C12N 5/10; A01H 5/00; A01H 5/10

(52) U.S. Cl. 800/289; 435/320.1; 435/419; 800/295; 800/278

(57) ABSTRACT

The present invention provides methods and compositions for producing transgenic plants having enhanced tolerance to biotic and/or abiotic stress and/or enhanced biomass production resulting from the expression of exogenous nucleotide sequences encoding SUMO E3 ligase or an active fragment thereof.

### STATEMENT OF PRIORITY

This application claims the benefit, under 35 U.S.C. §119(e), of U.S. Provisional Application No. 61/302,345, filed Feb. 8, 2010, the entire contents of which are incorporated by reference herein.

### STATEMENT OF GOVERNMENT SUPPORT

Aspects of this invention were funded under USDA-NIFA-BRAG Grant No. 2010-33522-21656. The U.S. Government has certain rights in this invention.

### FIELD OF THE INVENTION

The present invention relates to methods and compositions for producing transgenic plants with enhanced abiotic stress resistance and enhanced biomass production.

### BACKGROUND OF THE INVENTION

The need on a global scale for energy crops as renewable fuels and alternative sources of farm income is of great importance to current ecological and economic issues. The fast growing warm season perennial, switchgrass (Panicum virgantum L.), has been identified as an ideal candidate for biomass fuel production. Switchgrass use as a bioenergy feedstock, in addition to providing energy, might reduce net carbon gas emissions, improve soil and water quality, increase native wildlife habitat, and increase farm revenues. Optimizing plant biomass for increased production and enhancing plant adaptation to adverse environments play important roles in cost-effective use of bioenergy. Interrelated plant traits such as higher yield, and resilience to biotic and abiotic challenge will increase industrial crop value in terms of biofuels and biomaterials. Genetically engineered switchgrass with enhanced biomass production and plant tolerance to abiotic stresses can be directly used for commercialization, benefiting the environment and energy security.

Sumoylation regulates protein degradation and localization, protein-protein interaction, and transcriptional activity, impacting most cellular functions (Geiss-Friedlander and Melchior, 2007). SUMO conjugate levels increased when plants were subjected to a number of stresses, implicating sumoylation in plant stress responses (Kurepa et al., 2003; Lois et al., 2003).

Sumoylation is an essential mechanism of posttranslational modifications of proteins by the conjugation of small ubiquitin-related modifiers (SUMOs). It is a process of SUMO attachment to the substrate through formation of an isopeptide bond between the SUMO C-terminal Gly residue and the Lys residue located within a consensus motif of the target substrate, ΨKXE (Ψ is a hydrophobic amino acid, mostly Ilu, or Val, and X can be any residue). The sumoylation process begins with the activation of the SUMO C-terminal by an E1 activating enzyme, a subsequent transfer to a SUMO E2 conjugating enzyme, and then with the help of an E3 ligase, SUMO is finally conjugated to a substrate protein.

The SUMO E3 ligase plays a pivotal role in the sumoylation pathway. The SUMO E3 ligase SIZ1 from Arabidopsis has been demonstrated to be involved in regulation of plant growth, plant responses to phosphate starvation, water deficiency, cold and heat stresses, and salicylate-mediated innate immunity (Miura et al., 2005; Yoo et al., 2006; Catala et al., 2007; Lee et al., 2007; Miura et al., 2007).

The present invention addresses previous shortcomings in the art by providing methods and compositions for producing transgenic plants having enhanced tolerance to abiotic stress and/or enhanced biomass production resulting from the expression of exogenous nucleotide sequences encoding SUMO E3 ligase or an active fragment thereof.

### SUMMARY OF THE INVENTION

In one aspect, the present invention provides a nucleic acid construct comprising, in the following order from 5′ to 3′: a) a first promoter; b) a nucleotide sequence encoding small ubiquitin-related modifier (SUMO) E3 ligase or an active fragment thereof operably associated with the promoter of (a); c) a first termination sequence; d) a second promoter; e) a nucleotide sequence encoding a selectable marker operably associated with the promoter of (d); and f) a second termination sequence.

In a further aspect, the present invention provides a nucleic acid construct, comprising in the following order from 5′ to 3′: a) a corn ubiquitin promoter; b) a nucleotide sequence encoding rice SUMO E3 ligase; c) a first nos sequence; d) a CaMV 35S promoter; e) a nucleotide sequence encoding phosphinothricin acetyltransferase (bar); and f) a second nos sequence.

Further aspects of this invention include a transformed plant cell comprising the nucleic acid construct of this invention, a transgenic plant comprising the nucleic acid construct of this invention and/or the transformed plant cell of this invention, as well as a transgenic seed from the transgenic plant of this invention.

Additionally provided herein is a method of producing a transgenic plant having enhanced biomass production, comprising: a) transforming a cell of a plant with the nucleic acid construct of this invention; and b) regenerating the transgenic plant from the transformed plant cell, wherein the plant has enhanced biomass production as compared with a control plant that is not transformed with said nucleic acid construct.

Furthermore, the present invention provides a method of producing a transgenic plant having enhanced tolerance to biotic and/or abiotic stress, comprising: a) transforming a cell of a plant with the nucleic acid construct of this invention; and b) regenerating the transgenic plant from the transformed plant cell, wherein the plant has enhanced tolerance to biotic and/or abiotic stress as compared with a control plant that is not transformed with said nucleic acid construct.

In some embodiments of the methods of this invention, the biotic and/or abiotic stress can be: a) salt stress; b) drought stress; c) heat stress; d) oxidative stress; e) low temperature; f) flowering; g) phosphate deficiency; h) pathogen attack; i) abscisic acid signaling; j) salicylic acid signaling and k) any combination of (a)-(j) above. In particular embodiments, the stress is drought stress. In other particular embodiments, the stress is heat stress.

Furthermore, in the methods of this invention, the transgenic plant can have at least about 10%, about 20%, about 30%, about 40%, about 50% about 60%, about 70%, about 80%, about 90% or about 100% enhancement in biomass production as compared with the control plant.

Also in the methods of this invention directed to enhanced tolerance to a biotic and/or an abiotic stress, the transgenic plant can have about 10%, about 20%, about 30%, about 40%, about 50% about 60%, about 70%, about 80%, about 90% or about 100% enhancement in tolerance to said stress as compared with the control plant. In particular examples, the transgenic plant can have about 10%, about 20%, about 30%, about 40%, about 50% about 60%, about 70%, about 80%, about 90% or about 100% enhancement in tolerance to heat stress and/or drought stress as compared with the control plant.

Further provided herein is a transgenic plant produced by any of the methods of this invention, as well as a crop comprising a plurality of transgenic plants of this invention planted together in an agricultural field.

In various embodiments, the transgenic plant of this invention is turfgrass.

### BRIEF DESCRIPTION OF THE FIGURES

FIG. 1. Diagrams of the constructs pHL080, pHL080-1 and pHL080-2 of this invention. BR is the right border of transfer DNA (T-DNA); BL is the left border of T-DNA; and TP is transit peptide.

FIG. 2. Genomic DNA of OsSIZ1, a rice homologue of the Arabidopsis SIZ1 gene.

FIG. 3. Map of plasmid pHL080 and diagram of construct. The nucleotide sequence of pHL080 is provided herein as SEQ ID NO:13.

FIG. 4. Sequence alignment of two rice homologs of SUMO E3 ligase [OsSiz1 (SEQ ID NO:3) and OsSiz2 (SEQ ID NO:4)], a SUMO E3 ligase of Arabidopsis (AtSiz1, SEQ ID NO:1) and a SUMO E3 ligase of Medicago truncatula (MtSiz1, SEQ ID NO:2).

FIG. 5. Overexpression of a rice SUMO E3 ligase, OsSIZ1, leads to enhanced drought tolerance in transgenic turfgrass plants (WT=wild type; TG=transgenic).

FIG. 6. Overexpression of a rice SUMO E3 ligase, OsSIZ1, leads to enhanced heat tolerance in transgenic turfgrass plants (WT=wild type; TG=transgenic).

FIG. 7. Overexpression of the rice SUMO E3 ligase OsSIZ1 in transgenic turfgrass leads to enhanced plant growth. OsSIZ1-expressing transgenic (TG) creeping bentgrass plants exhibited greatly enhanced growth, producing significantly higher biomass than wild-type (WT) controls. OsSiz1-expressing transgenic creeping bentgrass plants exhibited better shoot growth. Transgenic (TG) and wild type (WT) plants initiated from individual stolons were grown in Elite 1200 Pots with pure sand and watered every three days with 200 ppm 20-10-20 fertilizer for 4 weeks. The clippings were collected for three weeks (4^{th}-6^{th} week) from trimmed-in-same-size of TG and WT plants. Asterisks (** or ***) indicate a significant difference between transgenic plants and wild-type controls at P<0.01 or 0.001, respectively, by student's t-test.

FIG. 8. A general strategy for controlled total vegetative growth in plants. Transgenic plants containing a construct in which the rice ubiquitin promoter and the RNAi construction or the antisense of the flower-specific gene, FLO/LFY homolog, is separated by the hyg gene flanked by directly oriented FRT sites will flower normally to produce seeds. When crossed to a plant expressing FLP recombinase, FLP should excise the blocking fragment (hyg gene), thus bringing together the ubiquitin promoter and the downstream antisense (left) or RNAi construct (right) of the FLO/LFY homolog gene, turning off the FLO/LFY homolog gene and giving rise to total vegetative growth in the hybrid.

FIG. 9. Semi-quantitative RT-PCR of OsSIZ1 gene in rice and turf tissues. 20-25 cycles of 95°/30S, 62°/30S, 72°/90S. L: 10 d old leaf; S: 10 d old seedling; R: 10 d old root; CS: carpel and stamen; F: flower; P1-6: 0.5, 5.0, 10, 15, 18, 20 cm panicle, respectively; WT: non-transgenic wild-type creeping bentgrass leaf; TG1: transgenic creeping bentgrass event 1; TG2: transgenic creeping bentgrass event 2. Rice tubulin (OsTua3) and Actin (OsActin1) genes were used as an internal control.

FIG. 10. Transgenic and wild type plants each deriving from a single stolon were grown in sand and trimmed carefully to the same size. Plants were watered daily with the basal nutrients (containing 1×MS micronutrients, 1/10× macronutrients without KH₂PO₄) supplemented with 1 μM KH₂PO₄. Wild-type (WT) plants exhibited typical phosphate deficiency symptom with an inhibited growth whereas transgenic plants (TG) showed much better performance. Transgenic and wild type plants each deriving from a single stolon were grown in sand and trimmed carefully to the same size. Plants were watered daily with the basal nutrients (containing 1×MS micronutrients, 1/10× macronutrients without KH₂PO₄) supplemented with 1 μM KH₂PO₄. Wild-type (WT) plants exhibited typical phosphate deficiency symptom with an inhibited growth whereas transgenic plants (TG) showed much better performance.

FIG. 11. Transgenic and wild type plants each deriving from a single stolon were grown in sand and trimmed carefully to the same size. Plants were watered daily with the basal nutrients (containing 1×MS micronutrients, 1/10× macronutrients without KH₂PO₄) supplemented with 1 μM KH₂PO₄. Wild-type (WT) plants exhibited typical phosphate deficiency symptoms with an inhibited growth whereas transgenic plants (TG) showed much better performance.

FIG. 12. Plant root phosphate content. Four replicates of both WT and TG plants in the Dillen cone-tainers were treated with 10 μM KH₂PO₄. Data are presented as means SD (n=4) and error bars represent SD. Asterisks (*, ** or ***) indicate a significant difference between transgenic plants and wild-type controls at P<0.05, 0.01, or 0.001, respectively, by student's t-test. TG plants exhibited enhanced phosphate uptake compared to WT controls.

FIG. 13. Plant leaf phosphate content. Four replicates of both WT and TG plants in the Dillen cone-tainers were treated with various concentrations of KH₂PO₄. Data are presented as means±SD (n=4) and error bars represent SD. Asterisks (*, ** or ***) indicate a significant difference between transgenic plants and wild-type controls at P<0.05, 0.01, or 0.001, respectively, by student's t-test. TG plants exhibited enhanced phosphate uptake compared to WT controls.

FIG. 14. Plant root potassium content. Four replicates of both WT and TG plants in the Dillen cone-tainers were treated with various concentrations of KH₂PO₄. Data are presented as means±SD (n=4) and error bars represent SD. Asterisks (*, ** or ***) indicate a significant difference between transgenic plants and wild-type controls at P<0.05, 0.01, or 0.001, respectively, by student's t-test. Compared to WT controls, TG plants exhibited enhanced root potassium uptake.

FIG. 15. Plant leaf potassium content. Four replicates of both WT and TG plants in the Dillen cone-tainers were treated with various concentrations of KH₂PO₄. Data are presented as means±SD (n=4) and error bars represent SD. Asterisks (*, ** or ***) indicate a significant difference between transgenic plants and wild-type controls at P<0.05, 0.01, or 0.001, respectively, by student's t-test. Compared to WT controls, TG plants exhibited increased leaf potassium content when lower concentration of phosphate was supplied.

### DETAILED DESCRIPTION OF THE INVENTION

The present invention will now be described more fully hereinafter with reference to the accompanying drawings and specification, in which preferred embodiments of the invention are shown. This invention may, however, be embodied in different forms and should not be construed as limited to the embodiments set forth herein.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

As used herein, “a,” “an” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of dose (e.g., an amount of a non-viral vector) and the like, is meant to encompass variations of ±20%, ±10%, ±5%, ±1%, ±0.5%, or even±0.1% of the specified amount.

As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP §2111.03. Thus, the term “consisting essentially of” when used in a claim of this invention is not intended to be interpreted to be equivalent to “comprising.”

The present invention is based on the discovery that the introduction into a plant of one or more of the nucleic acid constructs of this invention, which comprise nucleotide sequence(s) encoding a SUMO E3 ligase or an active fragment thereof, results in the production of a transgenic plant having increased or enhanced tolerance to biotic and/or abiotic stress and/or enhanced biomass production. The increase or enhancement in these plants is relative to the tolerance to biotic and/or abiotic stress and/or biomass production identified in a plant that does not comprise the nucleic acid construct(s) of this invention (i.e., a control plant).

Thus, in one embodiment, the present invention provides a nucleic acid construct comprising, consisting essentially of and/or consisting of one or more (e.g., 2, 3, 4, 5, 6, 7, 8, 9, 10, etc) nucleotide sequences encoding a SUMO E3 ligase or an active fragment thereof and operably associated with a promoter. In various embodiments, the SUMO E3 ligase or an active fragment thereof can be of plant origin or animal origin. The nucleic acid construct can comprise, consist essentially of and/or consist of a single nucleotide sequence encoding a SUMO E3 ligase or an active fragment thereof as well as multiple nucleotide sequences each encoding and/or all together encoding a SUMO E3 ligase or an active fragment thereof. The SUMO E3 ligase or an active fragment thereof can be combined on a single construct in any combination (e.g., SUMO E3 ligase(s) or active fragments thereof from any organism, in any order and in any combination of multiples).

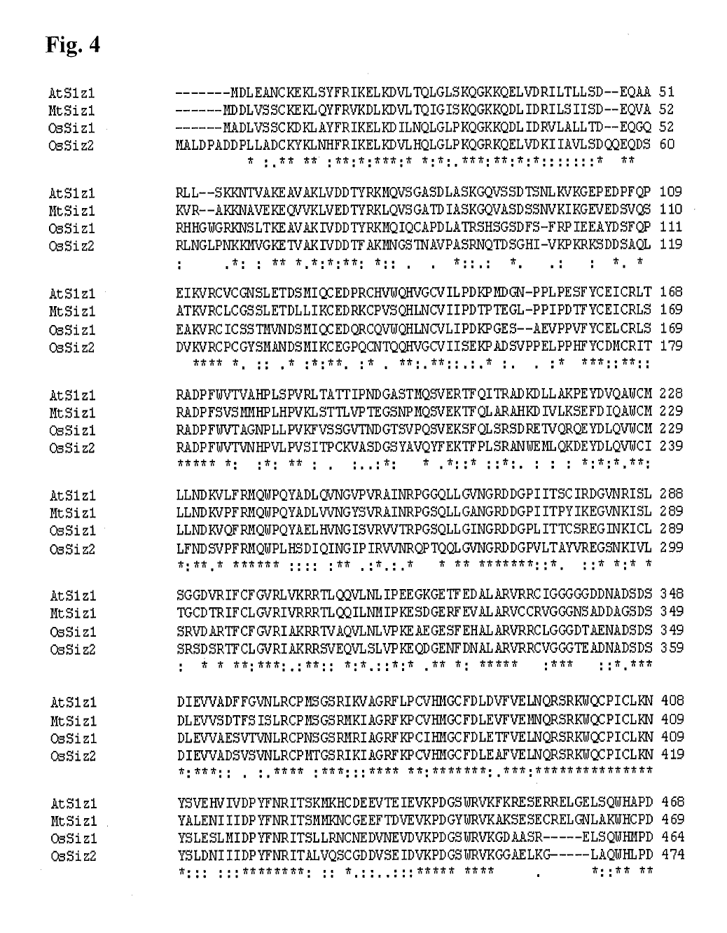

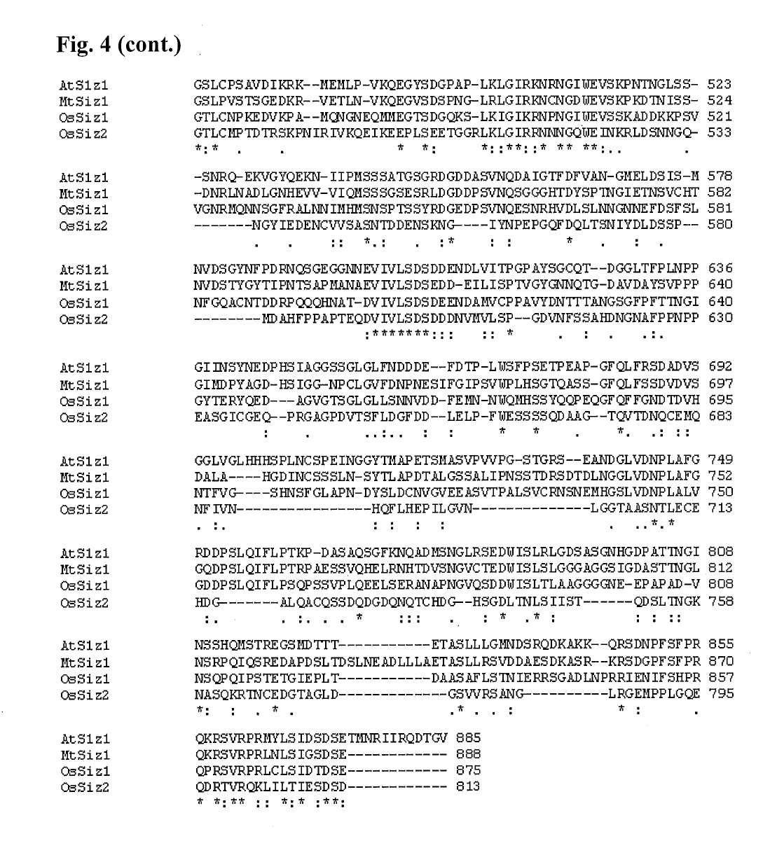

Nonlimiting examples of a SUMO E3 ligase of this invention include a SUMO E3 ligase from rice (e.g., Os05g0125000; GenBank® Database Accession Number NP_{—}001054517.1 (SEQ ID NO:3), encoded by GenBank® Database Accession Number NM_{—}001061052.1 (SEQ ID NO:15); Os03g0719100, GenBank® Database Accession Number NP_{—}001051092.1 (SEQ ID NO:4), encoded by GenBank® Database Accession Number NM_{—}001057627.1 (SEQ ID NO:16)), from sorghum (e.g., sorghum bicolor hypothetical protein; GenBank® Database Accession Number XP_{—}002439205.1 (SEQ ID NO:17), encoded by GenBank® Database Accession Number XM_{—}002439160.1, (SEQ ID NO:18)), from grape (e.g., Vitis vinifera hypothetical protein; GenBank® Database Accession Number XP_{—}002284945.1 (SEQ ID NO:19), encoded by GenBank® Database Accession Number XM_{—}002284909.1, (SEQ ID NO:20)), from Arabidopsis (e.g., Arabidopsis thaliana DNA binding/SUMO ligase (SIZ1); GenBank® Database Accession Number NP_{—}974969.1 (SEQ ID NO:1), encoded by GenBank® Database Accession Number NM_{—}203240.2 (SEQ ID NO:21)); from castor bean (e.g., Ricinus communis sumo ligase, putative; GenBank®Database Accession Number XP_{—}002526319.1 (SEQ ID NO:22), encoded by GenBank®Database Accession Number XM_{—}002526319.1 (SEQ ID NO:23)); and from legume (e.g., Medicago truncatula DNA-binding SAP; Zinc finger, MIZ-type; Zinc finger, FYVE/PHD-type; GenBank® Database Accession Number ABD33066 (SEQ ID NO:2)), see also TC120447 (SEQ ID NO:24) and TC114015 (SEQ ID NO:25), SEQ ID NO:2 is encoded by SEQ ID NO:25. The cDNA clone sequence of OsSiz1 is provided herein as SEQ ID NO:14. See also alignment of sequences of SEQ ID NOS:1-4 in FIG. 4.

The SUMO E3 ligase gene has three domains and one or more of these domains may be used in the constructs and methods of this invention to produce an active fragment of a SUME E3 ligase. The present invention also includes any fragment of the SUMO E3 ligase having biological activity, as well as the nucleotide sequence encoding such fragments. Thus, an active fragment of the SUMO E3 ligase of this invention can comprise amino acids at the amino terminus, amino acids at the carboxyl terminus and/or amino acids in the middle of the SUMO E3 ligase. A fragment of this invention can comprise, consist essentially of or consist of 10, 20, 30, 40, 50, 60, 70, 80, 90, 100, 125, 150, 175, 200, 250, 300, 350, 400, 450, 500, 550, 600, 650, 700, 750, 800 or 850 amino acids of the SUMO E3 ligase, which amino acids can be consecutive amino acids as well as a fusion of small fragments of consecutive amino acids with other small fragments of consecutive amino acids to produce a contiguous polypeptide. An active fragment of a SUMO E3 ligase is a fragment that can be demonstrated to have one or more of the known biological activities of the SUMO E3 ligase, as are well known in the art and as described herein. The production and testing of such fragments to identify those with a biological activity can be carried out according to protocols routine in the art.

These domains are (1) a MIZ/SP-RING zinc finger, (2) a SAP domain and (3) a PHD-finger. The MIZ/SP-RING zinc finger domain has SUMO (small ubiquitin-like modifier) ligase activity and is involved in DNA repair and chromosome organization. The SAP motif (after SAF-A/B, Acinus and PIAS) is a putative DNA/RNA binding domain found in diverse nuclear and cytoplasmic proteins. The PHD-finger folds into an interleaved type of Zn-finger chelating two Zn ions in a similar manner to that of the RING and FYVE domains. Several PHD fingers have been identified as binding modules of methylated histone H3.

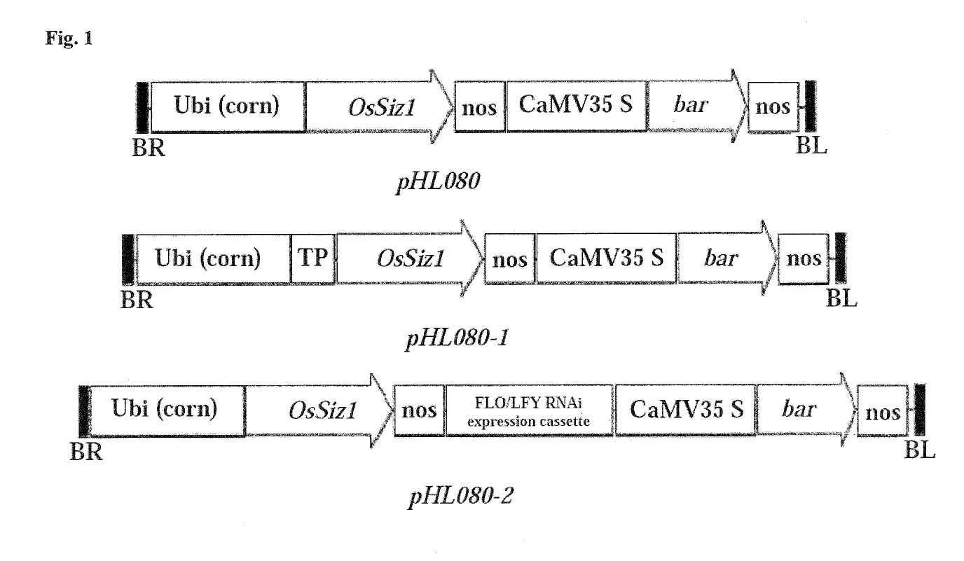

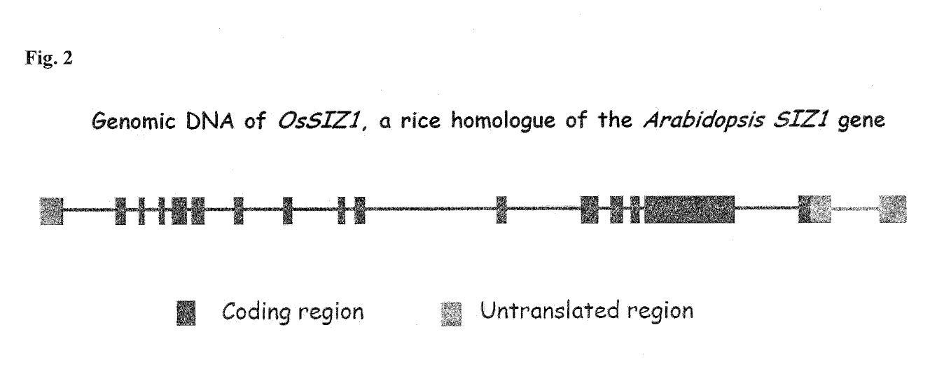

Various nonlimiting examples of a nucleic acid construct of this invention are provided in FIGS. 1, 2 and 3. Particular embodiments of this invention comprise, consist essentially of and/or consist of the following nucleic acid constructs.

A nucleic acid construct of this invention can comprising in the following order from 5′ to 3′: a) a first promoter; b) a nucleotide sequence encoding small ubiquitin-related modifier (SUMO) E3 ligase or an active fragment thereof operably associated with the promoter of (a); c) a first termination sequence; d) a second promoter; e) a nucleotide sequence encoding a selectable marker operably associated with the promoter of (d); and f) a second termination sequence.

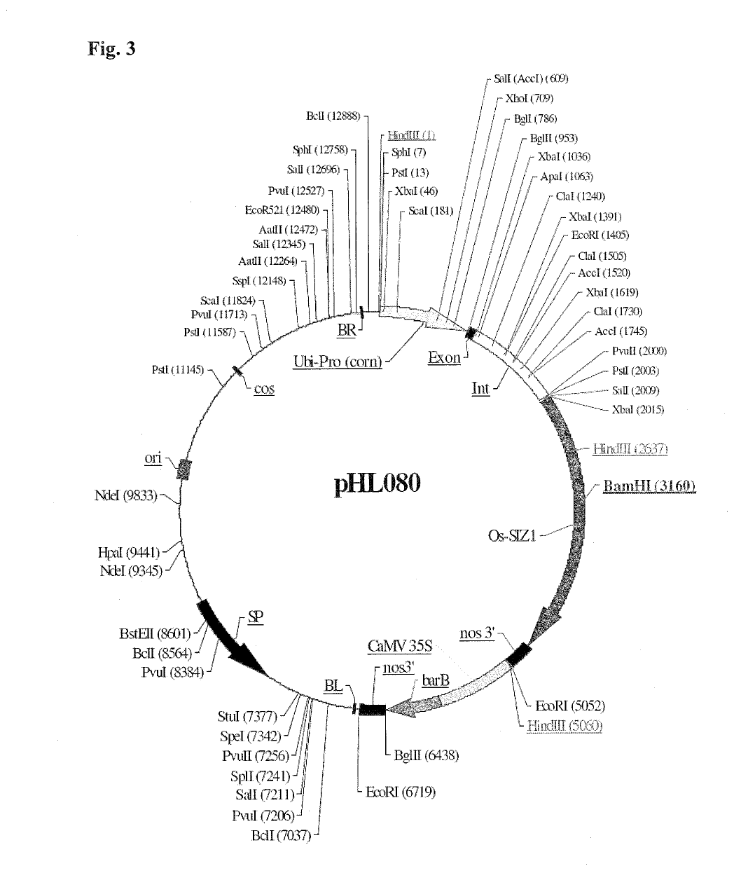

In various embodiments, the nucleic acid construct of this invention can comprise in the following order from 5′ to 3′: a) a corn ubiquitin promoter; b) a nucleotide sequence encoding rice SUMO E3 ligase or an active fragment thereof; c) a first nos sequence; d) a CaMV 35S promoter; e) a nucleotide sequence encoding phosphinothricin acetyltransferase (bar); and f) a second nos sequence. This construct is pHL080 in FIGS. 1 and 3.

In further embodiments, a nucleic acid construct of this invention can comprise in the following order from 5′ to 3′: a) a corn ubiquitin promoter; b) transit peptide (TP); c) a nucleotide sequence encoding rice SUMO E3 ligase or an active fragment thereof; d) a first nos sequence; e) a CaMV35S promoter; f) a nucleotide sequence encoding phosphinothricin acetyltransferase (bar); and g) a second nos sequence. This construct is pHL080-1 in FIG. 1.

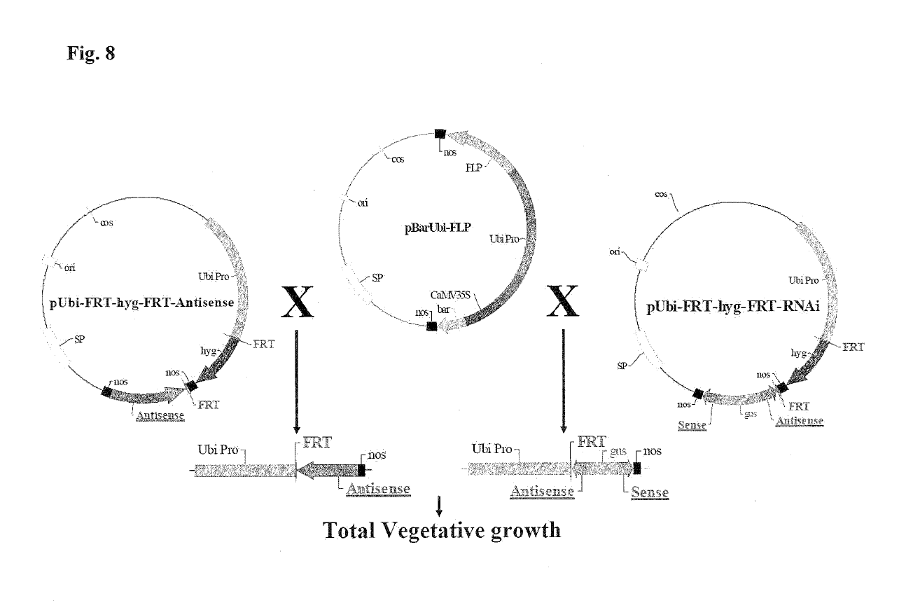

Also provided herein is a nucleic acid construct, comprising in the following order from 5′ to 3′: a) a corn ubiquitin promoter; b) a nucleotide sequence encoding rice SUMO E3 ligase or an active fragment thereof; c) a first nos sequence; d) a FLO/LFY RNAi expression cassette; e) a CaMV35S promoter; e) a nucleotide sequence encoding phosphinothricin acetyltransferase (bar); and f) a second nos sequence. This is the pHL080-2 construct in FIG. 1. An example of a general strategy for controlled total vegetative growth in plants is provided in FIG. 8.

The elements of the nucleic acid constructs of the present invention can be in any combination. Thus, in the nucleic acid constructs described above, with the elements defined as being in the order listed, the respective elements can be present in the order described and immediately adjacent to the next element upstream and/or downstream, with no intervening elements and/or the respective elements can be present in the order described and intervening elements can be present between the elements, in any combination.

In addition, in nucleic acid constructs of this invention that comprise multiples of the same type of element (e.g., a first promoter and a second promoter or a first termination sequence and a second termination sequence or a first nucleotide sequence encoding a SUMO Ed ligase or active fragment thereof and a second nucleotide sequence encoding a SUMO E3 ligase or active fragment thereof) in a single construct, such similarly named elements can be the same or they can be different in any combination (e.g., a first promoter sequence can be a corn ubiquitin promoter sequence and a second promoter sequence can be rice ubiquitin promoter sequence or a first termination sequence can be nos and a second termination sequence can also be nos, etc.).

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment, genomic DNA, synthetic (e.g., chemically synthesized) DNA, plasmid DNA, mRNA, and anti-sense RNA, any of which can be single stranded or double stranded. The terms “nucleotide sequence,” “nucleic acid,” “nucleic acid molecule,” “oligonucleotide” and “polynucleotide” are also used interchangeably herein to refer to a heteropolymer of nucleotides.

Nucleic acids of this invention can comprise a nucleotide sequence that can be identical in sequence to the sequence which is naturally occurring or, due to the well-characterized degeneracy of the nucleic acid code, can include alternative codons that encode the same amino acid as that which is found in the naturally occurring sequence. Furthermore, nucleic acids of this invention can comprise nucleotide sequences that can include codons which represent conservative substitutions of amino acids as are well known in the art, such that the biological activity of the resulting polypeptide and/or fragment is retained. A nucleic acid of this invention can be single or double stranded. Additionally, the nucleic acids of this invention can also include a nucleic acid strand that is partially complementary to a part of the nucleic acid sequence or completely complementary across the full length of the nucleic acid sequence. Nucleic acid sequences provided herein are presented herein in the 5′ to 3′ direction, from left to right and are represented using the standard code for representing the nucleotide characters as set forth in the U.S. sequence rules, 37 CFR §§1.821-1.825 and the World Intellectual Property Organization (WIPO) Standard ST.25.

As used herein, the term “gene” refers to a nucleic acid molecule capable of being used to produce mRNA or antisense RNA. Genes may or may not be capable of being used to produce a functional protein. Genes include both coding and non-coding regions (e.g., introns, regulatory elements, promoters, enhancers, termination sequences and 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

An “isolated” nucleic acid of the present invention is generally free of nucleic acid sequences that flank the nucleic acid of interest in the genomic DNA of the organism from which the nucleic acid was derived (such as coding sequences present at the 5′ or 3′ ends). However, the nucleic acid of this invention can include some additional bases or moieties that do not deleteriously affect the basic structural and/or functional characteristics of the nucleic acid. “Isolated” does not mean that the preparation is technically pure (homogeneous).

The term “transgene” as used herein, refers to any nucleic acid sequence used in the transformation of a cell or cells of a plant or other organism. Thus, a transgene can be a coding sequence, a non-coding sequence, a cDNA, a gene or fragment or portion thereof, a genomic sequence, a regulatory element and the like. A “transgenic” organism, such as a transgenic plant or transgenic animal, is an organism comprising cells into which a transgene has been delivered or introduced and the transgene can be expressed in the cells of the transgenic organism to produce a product, the presence of which can impart an effect (e.g., a therapeutic, beneficial and/or desirable effect) and/or a phenotype (e.g., a beneficial and/or desirable phenotype) in the organism.

As used herein, the term “promoter” refers to a region of a nucleotide sequence that incorporates the necessary signals for the efficient expression of a coding sequence. This may include sequences to which an RNA polymerase binds, but is not limited to such sequences and can include regions to which other regulatory proteins bind together with regions involved in the control of protein translation and can also include coding sequences.

Furthermore, a “plant promoter” of this invention is a promoter capable of initiating transcription in plant cells. Such promoters include those that drive expression of a nucleotide sequence constitutively, those that drive expression when induced, and those that drive expression in a tissue- or developmentally-specific manner, as these various types of promoters are known in the art.

Thus, for example, in some embodiments of the invention, a constitutive promoter can be used to drive the expression of a transgene of this invention in a plant cell. A constitutive promoter is an unregulated promoter that allows for continual transcription of its associated gene or coding sequence. Thus, constitutive promoters are generally active under most environmental conditions, in most or all cell types and in most or all states of development or cell differentiation.

Any constitutive promoter functional in a plant can be utilized in the instant invention. Exemplary constitutive promoters include, but are not limited to, the promoters from plant viruses including, but not limited to, the 35S promoter from CaMV (Odell et al., Nature 313: 810 (1985)); figwort mosaic virus (FMV) 35S promoter (P-FMV35S, U.S. Pat. Nos. 6,051,753 and 6,018,100); the enhanced CaMV35S promoter (e35S); the 1′- or 2′-promoter derived from T-DNA of Agrobacterium tumefaciens; the nopaline synthase (NOS) and/or octopine synthase (OCS) promoters, which are carried on tumor-inducing plasmids of Agrobacterium tumefaciens (Ebert et al., Proc. Natl. Acad. Sci. (U.S.A.), 84:5745 5749, 1987); actin promoters including, but not limited to, rice actin (McElroy et al., Plant Cell 2: 163 (1990); U.S. Pat. No. 5,641,876); histone promoters; tubulin promoters; ubiquitin and polyubiquitin promoters, including a corn ubiquitin promoter or a rice ubiquitin promoter ((Sun and Callis, Plant J, 11(5):1017-1027 (1997)); Christensen et al., Plant Mol. Biol. 12: 619 (1989) and Christensen et al., Plant Mol. Biol. 18: 675 (1992)); pEMU (Last et al., Theor. Appl. Genet. 81: 581 (1991)); the mannopine synthase promoter (MAS) (Velten et al., EMBO J. 3: 2723 (1984)); maize H3 histone promoter (Lepelit et al., Mol. Gen. Genet. 231: 276 (1992) and Atanassova et al., Plant Journal 2: 291 (1992)); the ALS promoter, a XbaI/NcoI fragment 5′ to the Brassica napus ALS3 structural gene (or a nucleotide sequence that has substantial sequence similarity to said XbaI/NcoI fragment); ACT11 from Arabidopsis (Huang et al., Plant Mol. Biol. 33:125-139 (1996)); Cat3 from Arabidopsis (GenBank No. U43147, Zhong et al., Mol. Gen. Genet. 251:196-203 (1996)); GPc1 from maize (GenBank No. X15596, Martinez et al., J. Mol. Biol. 208:551-565 (1989)); and Gpc2 from maize (GenBank No. U45855, Manjunath et al., Plant Mol. Biol. 33:97-112 (1997)), including any combination thereof.

In some embodiments of the present invention, an inducible promoter can be used to drive the expression of a transgene. Inducible promoters activate or initiate expression only after exposure to, or contact with, an inducing agent. Inducing agents include, but are not limited to, various environmental conditions (e.g., pH, temperature), proteins and chemicals. Examples of environmental conditions that can affect transcription by inducible promoters include pathogen attack, anaerobic conditions, extreme temperature and/or the presence of light. Examples of chemical inducing agents include, but are not limited to, herbicides, antibiotics, ethanol, plant hormones and steroids. Any inducible promoter that is functional in a plant can be used in the instant invention (see, Ward et al., (1993) Plant Mol. Biol. 22: 361 (1993)). Exemplary inducible promoters include, but are not limited to, promoters from the ACEI system, which respond to copper (Melt et al., PNAS 90: 4567 (1993)); the ln2 gene from maize, which responds to benzenesulfonamide herbicide safeners (Hershey et al., (1991) Mol. Gen. Genetics 227: 229 (1991) and Gatz et al., Mol. Gen. Genetics 243: 32 (1994)); a heat shock promoter, including, but not limited to, the soybean heat shock promoters Gmhsp 17.5-E, Gmhsp 17.2-E and Gmhsp 17.6-L and those described in U.S. Pat. No. 5,447,858; the Tet repressor from Tn10 (Gatz et al., Mol. Gen. Genet. 227: 229 (1991)) and the light-inducible promoter from the small subunit of ribulose bisphosphate carboxylase (ssRUBISCO), including any combination thereof. Other examples of inducible promoters include, but are not limited to, those described by Moore et al. (Plant J. 45:651-683 (2006)). Additionally, some inducible promoters respond to an inducing agent to which plants do not normally respond. An example of such an inducible promoter is the inducible promoter from a steroid hormone gene, the transcriptional activity of which is induced by a glucocorticosteroid hormone (Schena et al., Proc. Natl. Acad. Sci. U.S.A. 88: 421 (1991)).

In further embodiments of the present invention, a tissue-specific promoter can be used to drive the expression of a transgene in a particular tissue in the transgenic plant. Tissue-specific promoters drive expression of a nucleic acid only in certain tissues or cell types, e.g., in the case of plants, in the leaves, stems, flowers and their various parts, roots, fruits and/or seeds, etc. Thus, plants transformed with a nucleic acid of interest operably linked to a tissue-specific promoter produce the product encoded by the transgene exclusively, or preferentially, in a specific tissue or cell type.

Any plant tissue-specific promoter can be utilized in the instant invention. Exemplary tissue-specific promoters include, but are not limited to, a root-specific promoter, such as that from the phaseolin gene (Murai et al., Science 23: 476 (1983) and Sengupta-Gopalan et al., Proc. Natl. Acad. Sci. USA 82: 3320 (1985)); a leaf-specific and light-induced promoter such as that from cab or rubisco (Simpson et al. EMBO J. 4: 2723 (1985) and Timko et al., Nature 318: 579 (1985)); the fruit-specific E8 promoter from tomato (Lincoln et al. Proc. Nat'l. Acad. Sci. USA 84: 2793-2797 (1988); Deikman et al. EMBO J. 7: 3315-3320 (1988); Deikman et al. Plant Physiol. 100: 2013-2017 (1992); seed-specific promoters of, for example, Arabidopsis thaliana (Krebbers et al. (1988) Plant Physiol. 87:859); an anther-specific promoter such as that from LAT52 (Twell et al. Mol. Gen. Genet. 217: 240 (1989)) or European Patent Application No 344029, and those described by Xu et al. (Plant Cell Rep. 25:231-240 (2006)) and Gomez et al. (Planta 219:967-981 (2004)); a pollen-specific promoter such as that from Zm13 (Guerrero et al., Mol. Gen. Genet. 224: 161 (1993)), and those described by Yamaji et al. (Plant Cell Rep. 25:749-57 (2006)) and Okada et al. (Plant Cell Physiol. 46:749-802 (2005)); a pith-specific promoter, such as the promoter isolated from a plant TrpA gene as described in International PCT Publication No. WO93/07278; and a microspore-specific promoter such as that from apg (Twell et al. Sex. Plant Reprod. 6: 217 (1993)). Exemplary green tissue-specific promoters include the maize phosphoenol pyruvate carboxylase (PEPC) promoter, small subunit ribulose bis-carboxylase promoters (ssRUBISCO) and the chlorophyll a/b binding protein promoters, including any combination thereof.

A promoter of the present invention can also be developmentally specific in that it drives expression during a particular “developmental phase” of the plant. Thus, such a promoter is capable of directing selective expression of a nucleotide sequence of interest at a particular period or phase in the life of a plant (e.g., seed formation), compared to the relative absence of expression of the same nucleotide sequence of interest in a different phase (e.g. seed germination). For example, in plants, seed-specific promoters are typically active during the development of seeds and germination promoters are typically active during germination of the seeds. Any developmentally-specific promoter capable of functioning in a plant can be used in the present invention.

The nucleic acid construct of this invention can further comprise a termination sequence. Nonlimiting examples of a termination sequence of this invention include the nopaline synthase (nos) sequence (see, e.g., FIG. 1), gene 7 poly(A) signal, and CaMV 35S gene poly(A) signal.

The nucleic acid construct of this invention can further comprise a signal peptide sequence. Signal peptides may also be called targeting signals, transit peptides or localization signals. A signal or transit peptide contains a signal to direct (target) the whole protein to a particular subcellular compartment. Upon targeting to its destination, the signal peptide is cleaved, resulting in a mature protein product. Non limiting examples of transit peptides of this invention include peptides for chloroplast targeting and/or mitochondrial targeting.

An example of a transit peptide (TP) that can be used, e.g., in the constructs shown in FIG. 1 is MAPSVMASSATTVAPFQGLKSTAGMPVARRSGNSSFGNVSNGGRIRCM (SEQ ID NO:5), which is the first 48 amino acids of rice (Oryza sativa) ribulose bisphosphate carboxylase small chain (Accession No. NM_{—}001073091.1), located in the plastid of rice, that means the fused protein with this TP will be delivered to the plastid.

Other nonlimiting examples of a signal peptide sequence of this invention include the signal sequence of the tobacco AP24 protein (Coca et al. 2004); the signal peptide of divergicin A (Worobo et al. 1995); the proteinase inhibitor II signal peptide (Herbers et al. 1995); and the signal peptide from a Coix prolamin (Leite et al. 2000, Ottoboni et al. (1993), including any combination thereof.

The nucleic acid construct of this invention can further comprise a linker peptide. Nonlimiting examples of a linker peptide of this invention include the IbAMP propeptide (Francois et al. 2002, Sabelle et al. 2002); the 2A sequence of foot and mouth disease virus (Ma et al. 2002); a GUS linker peptide, and a serine rich peptide linker [e.g., Ser, Ser, Ser, Ser, Gly)_{y} where y≧1 (U.S. Pat. No. 5,525,491), including any combination thereof.

The nucleic acid constructs of the present invention can further comprise a nucleotide sequence encoding a selectable marker, operably linked to a regulatory element (a promoter, for example) that allows transformed cells in which the expression product of the selectable marker sequence is produced, to be recovered by either negative selection, i.e., inhibiting growth of cells that do not contain the selectable marker, or positive selection, i.e., screening for the product encoded by the selectable marker coding sequence. For example, in one embodiment the nucleic acid construct can comprise a phosphinothricin acetyltransferase (bar) coding sequence operably associated with a rice ubiquitin promoter sequence.

Many commonly used selectable marker coding sequences for plant transformation are well known in the transformation art, and include, for example, nucleotide sequences that code for enzymes that metabolically detoxify a selective chemical agent which may be an antibiotic or a herbicide, and/or nucleotide sequences that encode an altered target which is insensitive to the inhibitor (See e.g., Aragão et al., Braz. J Plant Physiol. 14: 1-10 (2002)). Any nucleotide sequence encoding a selectable marker that can be expressed in a plant is useful in the present invention.

One commonly used selectable marker coding sequence for plant transformation is the nucleotide sequence encoding neomycin phosphotransferase II (npfII), isolated from transposon Tn5, which when placed under the control of plant regulatory signals confers resistance to kanamycin (Fraley et al., Proc. Natl. Acad. Sci. USA., 80: 4803 (1983)). Another commonly used selectable marker coding sequence encodes hygromycin phosphotransferase, which confers resistance to the antibiotic hygromycin (Vanden Elzen et al., Plant Mol. Biol., 5: 299 (1985)).

Some selectable marker coding sequences confer resistance to herbicides. Herbicide resistance sequences generally encode a modified target protein insensitive to the herbicide or an enzyme that degrades or detoxifies the herbicide in the plant before it can act (DeBlock et al., EMBO J. 6, 2513 (1987); DeBlock et al., Plant Physiol. 91, 691 (1989); Fromm et al., BioTechnology 8, 833 (1990); Gordon-Kamm et al., Plant Cell 2, 603 (1990)). For example, resistance to glyphosphate or sulfonylurea herbicides has been obtained using marker sequences coding for the mutant target enzymes, 5-enolpyruvylshikimate-3-phosphate synthase (EPSPS) and acetolactate synthase (ALS). Resistance to glufosinate ammonium, boromoxynil, and 2,4-dichlorophenoxyacetate (2,4-D) has been obtained by using bacterial nucleotide sequences encoding phosphinothricin acetyltransferase, a nitrilase, or a 2,4-dichlorophenoxyacetate monooxygenase, which detoxify the respective herbicides.

Other selectable marker coding sequences for plant transformation are not of bacterial origin. These coding sequences include, for example, mouse dihydrofolate reductase, plant 5-eno/pyruvylshikimate-3-phosphate synthase and plant acetolactate synthase (Eichholtz et al., Somatic Cell Mol. Genet. 13: 67 (1987); Shah et al., Science 233: 478 (1986); Charest et al., Plant Cell Rep. 8: 643 (1990)).

Another class of marker coding sequences for plant transformation requires screening of presumptively transformed plant cells rather than direct genetic selection of transformed cells for resistance to a toxic substance such as an antibiotic. These coding sequences are particularly useful to quantify or visualize the spatial pattern of expression of a nucleotide sequence in specific tissues and are frequently referred to as reporter nucleotide sequences because they can be fused to a gene or gene regulatory sequence for the investigation of gene expression. Commonly used nucleotide sequences for screening presumptively transformed cells include, but are not limited to, those encoding β-glucuronidase (GUS), β-galactosidase, luciferase and chloramphenicol acetyltransferase (Jefferson Plant Mol. Biol. Rep. 5:387 (1987); Teen et al. EMBO J. 8:343 (1989); Koncz et al. Proc. Natl. Acad. Sci. U.S.A. 84:131 (1987); De Block et al. EMBO J. 3:1681 (1984)).

Some in vivo methods for detecting GUS activity that do not require destruction of plant tissue are available (e.g., Molecular Probes Publication 2908, Imagene Green™, p. 1-4 (1993) and Naleway et al., J. Cell Biol. 115:15 (1991)). In addition, a nucleotide sequence encoding green fluorescent protein (GFP) has been utilized as a marker for expression in prokaryotic and eukaryotic cells (Chalfie et al., Science 263:802 (1994)). GFP and mutants of GFP may be used as screenable markers. Similar to GFP, red fluorescent protein, (DsRed2) has also been used as a selectable marker in plants (Nishizawa et al., Plant Cell Reports 25 (12): 1355-1361 (2006)). In addition, reef coral proteins have been used as selectable markers in plants (Wenck et al. Plant Cell Reports 22(4):244-251 (2003)).

For purposes of the present invention, selectable marker coding sequences can also include, but are not limited to, nucleotide sequences encoding: neomycin phosphotransferase I and II (Southern et al., J. Mol. Appl. Gen. 1:327 (1982)); Fraley et al., CRC Critical Reviews in Plant Science 4:1 (1986)); cyanamide hydratase (Maier-Greiner et al., Proc. Natl. Acad. Sci. USA 88:4250 (1991)); aspartate kinase; dihydrodipicolinate synthase (Peri et al., BioTechnology 11, 715 (1993)); bar gene (Told et al., Plant Physiol. 100:1503 (1992); Meagher et al., Crop Sci. 36:1367 (1996)); tryptophane decarboxylase (Goddijn et al., Plant Mol. Biol. 22:907 (1993)); hygromycin phosphotransferase (HPT or HYG; Shimizu et al., Mol. Cell. Biol. 6:1074 (1986); Waldron et al., Plant Mol. Biol. 5:103 (1985); Zhijian et al., Plant Science 108:219 (1995)); dihydrofolate reductase (DHFR; Kwok et al., Proc. Natl. Acad. Sci. USA 83:4552 (1986)); phosphinothricin acetyltransferase (DeBlock et al., EMBO J. 6:2513 (1987)); 2,2-dichloropropionic acid dehalogenase (Buchanan-Wollatron et al., J. Cell. Biochem. 13D:330 (1989)); acetohydroxyacid synthase (U.S. Pat. No. 4,761,373 to Anderson et al.; Haughn et al., Mol. Gen. Genet. 221:266 (1988)); 5-enolpyruvyl-shikimate-phosphate synthase (aroA; Comai et al., Nature 317:741 (1985)); haloarylnitrilase (PCT Publication No. WO 87/04181 to Stalker et al.); acetyl-coenzyme A carboxylase (Parker et al., Plant Physiol. 92:1220 (1990)); dihydropteroate synthase (sulI; Guerineau et al., Plant Mol. Biol. 15:127 (1990)); and 32 kDa photosystem II polypeptide (psbA; Hirschberg et al., Science 222:1346 (1983)).

Also included are nucleotide sequences that encode polypeptides that confer resistance to: gentamicin (Miki et al., J. Biotechnol. 107:193-232 (2004)); chloramphenicol (Herrera-Estrella et al., EMBO J. 2:987 (1983)); methotrexate (Herrera-Estrella et al., Nature 303:209 (1983); Meijer et al., Plant Mol. Biol. 16:807 (1991)); Meijer et al., Plant Mol. Bio. 16:807 (1991)); streptomycin (Jones et al., Mol. Gen. Genet. 210:86 (1987)); spectinomycin (Bretagne-Sagnard et al., Transgenic Res. 5:131 (1996)); bleomycin (Hille et al., Plant Mol. Biol. 7, 171 (1986)); sulfonamide (Guerineau et al., Plant Mol. Bio. 15:127 (1990); bromoxynil (Stalker et al., Science 242:419 (1988)); 2,4-D (Streber et al., Bio/Technology 7, 811 (1989)); phosphinothricin (DeBlock et al., EMBO J. 6:2513 (1987)); and/or spectinomycin (Bretagne-Sagnard and Chupeau, Transgenic Research 5:131 (1996)).

The product of the bar gene confers herbicide resistance to glufosinate-type herbicides, such as phosphinothricin (PPT) or bialaphos, and the like. As noted above, other selectable markers that could be used in the nucleic acid constructs of the present invention include, but are not limited to, the pat gene or coding sequence, the expression of which also confers resistance to bialaphos and phosphinothricin resistance, the ALS gene or coding sequence for imidazolinone resistance, the HPH or HYG gene or coding sequence for hygromycin resistance (Coca et al. 2004), the EPSP synthase gene or coding sequence for glyphosate resistance, the Hm1 gene or coding sequence for resistance to the Hc-toxin, a coding sequence for streptomycin phosphotransferase resistance (Mazodier et al.) and/or other selective agents used routinely and known to one of ordinary skill in the art. See generally, Yarranton, Curr. Opin. Biotech. 3:506 (1992); Chistopherson et al., Proc. Natl. Acad. Sci. USA 89:6314 (1992); Yao et al., Cell 71:63 (1992); Reznikoff, Mol. Microbiol. 6:2419 (1992); Barkley et al., The Operon 177-220 (1980); Hu et al., Cell 48:555 (1987); Brown et al., Cell 49:603 (1987); Figge et al., Cell 52:713 (1988); Deuschle et al., Proc. Natl. Acad. Sci. USA 86:400 (1989); Fuerst et al., Proc. Natl. Acad. Sci. USA 86:2549 (1989); Deuschle et al., Science 248:480 (1990); Labow et al., Mol. Cell. Biol. 10:3343 (1990); Zambretti et al., Proc. Natl. Acad. Sci. USA 89:3952 (1992); Bairn et al., Proc. Natl. Acad. Sci. USA 88:5072 (1991); Wyborski et al., Nuc. Acids Res. 19:4647 (1991); Hillenand-Wissman, Topics in Mol. And Struc. Biol. 10:143 (1989); Degenkolb et al., Antimicrob. Agents Chemother. 35:1591 (1991); Kleinschnidt et al., Biochemistry 27:1094 (1988); Gatz et al., Plant J. 2:397 (1992); Gossen et al., Proc. Natl. Acad. Sci. USA 89:5547 (1992); Oliva et al., Antimicrob. Agents Chemother. 36:913 (1992); Hlavka et al., Handbook of Experimental Pharmacology 78 (1985); and Gill et al., Nature 334:721 (1988). A review of approximately 50 marker genes in transgenic plants is provided in Miki et al. (2003), the entire contents of which are incorporated by reference herein.

Additionally, for purposes of the present invention, selectable markers include nucleotide sequence(s) conferring environmental or artificial stress resistance or tolerance including, but not limited to, a nucleotide sequence conferring high glucose tolerance, a nucleotide sequence conferring low phosphate tolerance, a nucleotide sequence conferring mannose tolerance, and/or a nucleotide sequence conferring drought tolerance, salt tolerance or cold tolerance. Examples of nucleotide sequences that confer environmental or artificial stress resistance or tolerance include, but are not limited to, a nucleotide sequence encoding trehalose phosphate synthase, a nucleotide sequence encoding phophomannose isomerase (Negrotto et al., Plant Cell Reports 19(8):798-803 (2003)), a nucleotide sequence encoding the Arabidopsis vacuolar H⁺-pyrophosphatase gene, AVP1, a nucleotide sequence conferring aldehyde resistance (U.S. Pat. No. 5,633,153), a nucleotide sequence conferring cyanamide resistance (Weeks et al., Crop Sci 40:1749-1754 (2000)) and those described by Iuchi et al. (Plant J. 27(4):325-332 (2001)); Umezawa et al. (Curr Opin Biotechnol. 17(2):113-22 (2006)); U.S. Pat. No. 5,837,545; Oraby et al. (Crop Sci. 45:2218-2227 (2005)) and Shi et al. (Proc. Natl. Acad. Sci. 97:6896-6901 (2000)).

The above list of selectable marker genes and coding sequences is not meant to be limiting as any selectable marker coding sequence now known or later identified can be used in the present invention. Also, a selectable marker of this invention can be used in any combination with any other selectable marker.

In some embodiments of this invention, the nucleic acid construct of this invention can comprise gene elements to control gene flow in the environment in which a transgenic plant of this invention could be placed. Examples of such elements are described in International Publication No. WO 2009/011863, the disclosures of which are incorporated by reference herein.

In some embodiments, the nucleic acid construct of this invention can comprise elements to impart sterility to the transgenic plant into which the nucleic acid construct is introduced in order to control movement of the transgene(s) of this invention in the environment. As one example, RNAi technology can be used to turn off the expression of certain endogenous genes, resulting in a plant that maintains vegetative growth during its whole life cycle. RNAi technology to knock out the FLO/LFY homolog gene, achieving total sterility in transgenic plants, can be used in combination with the overexpression of OsSIZ1 to produce environmentally safe transgenic plants (e.g., perennials) with enhanced performance as described herein. An example of a nucleic acid construct (pHL080-2) of this invention comprising a FLO/LFY RNAi expression cassette is shown in FIG. 1.

In other embodiments, a flower-specific or pollen-specific promoter can be used to drive cytotoxic genes, such as the ribonuclease gene, barnase; or the RNAi of any other genes essential for flower or pollen development to achieve total sterility or male sterility for transgene containment. Inducible promoters or a site-specific recombination system can also be used to achieve controlled male sterility or total sterility for gene containment.

Elements that can impart sterility to the transgenic plant include, but are not limited to, nucleotide sequences, or fragments thereof, that modulate the reproductive transition from a vegetative meristem or flower promotion gene or coding sequence, or flower repressor gene or coding sequence. Three growth phases are generally observed in the life cycle of a flowering plant: vegetative, inflorescence and floral. The switch from vegetative to reproductive or floral growth requires a change in the developmental program of the descendents of the stem cells in the shoot apical meristem. In the vegetative phase, the shoot apical meristem generates leaves that provide resources necessary to produce fertile offspring. Upon receiving the appropriate environmental and developmental signals, the plant switches to floral (reproductive) growth and the shoot apical meristem enters the inflorescence phase, giving rise to an inflorescence with flower primordia. During this phase, the fate of the shoot apical meristem and the secondary shoots that arise in the axils of the leaves is determined by a set of meristem identity genes, some of which prevent and some of which promote the development of floral meristems. Once established, the plant enters the late inflorescence phase where the floral organs are produced. Two basic types of inflorescence have been identified in plants: determinate and indeterminate. In a species producing a determinate inflorescence, the shoot apical meristem eventually produces floral organs and the production of meristems is terminated with a flower. In those species producing an indeterminate inflorescence, the shoot apical meristem is not converted to a floral identity and therefore only produces floral meristems from its periphery, resulting in a continuous growth pattern.

In dicots, after the transition from vegetative to reproductive development, floral meristems are initiated by the action of a set of genes called floral meristem identity genes. FLORICAULA (flo) of Antirrhinum and its Arabidopsis counterpart, LEAFY (lfy), are floral meristem identity genes that participate in the reproductive transition to establish floral fate. In strong flo and lfy mutant plants, flowers are transformed into inflorescence shoots (Coen et al., Cell 63:1311-1322 (1990); Weigel et al. Cell 69:843-859, (1992)), indicating that flo and lfy are exemplary flower-promotion genes.

In monocots, FLO/LFY homologs have been identified in several species, such as rice (Kyozuka et al., Proc. Natl. Acad. Sci. 95:1979-1982 (1998)); Lolium temulentum, maize, and ryegrass (Lolium perenne). The FLO/LFY homologs from different species have high amino acid sequence homology and are well conserved in the C-terminal regions (Kyozuka et al., Proc. Natl. Acad. Sci. 95:1979-1982 (1998); Bomblies et al., Development 130:2385-2395 (2003)).

In addition to flo/lfy genes or coding sequences, other examples of flower promotion genes or coding sequences include, but are not limited to, APETALA1 (Accession no. NM105581)/SQUAMOSA (ap1/squa) in Arabidopsis and Antirrhinum, CAULIFLOWER (cal, Accession no. AY174609), FRUITFUL (ful, Accession no. AY173056), FLOWERING LOCUS T (Accession no. AB027505), and SUPPRESSOR OF OVEREXPRESSION OF CONSTANS1 (soc1) in Arabidopsis (Samach et al., Science 288:1613-1616 (2000); Simpson and Dean, Science 296:285-289 (2002)); Zik et al., Annu. Rev. Cell Dev. Biol. 19:119-140 (2003)).

Additional non-limiting examples of flowering related genes or coding sequences include TERMINAL FLOWER 1 (tfl1) in Arabidopsis and its homolog CENTRORADIALS (cen) in Antirrhinum; FLOWERING LOCUS C (flc) and the emf gene in Arabidopsis. It is noted that any flower-promotion or flower-related coding sequence(s), the down-regulation of which results in no or reduced sexual reproduction (or total vegetative growth), can be used in the present invention.

Down-regulation of expression of one or more flower promotion or coding sequences in a plant, such as a flo/lfy homolog, results in reduced or no sexual reproduction or total vegetative growth in the transgenic plant, whereby the transgenic plant is unable to produce flowers (or there is a significant delay in flower production). The high conservation observed among flo/lfy homologs indicates that further flo/lfy homologs can be isolated from other plant species by using, for example, the methods of Kyozuka et al. (Proc. Natl. Acad. Sci. 95:1979-1982 (1998)) and Bomblies et al. (Development 130:2385-2395 (2003)). For example, the flo/lfy homolog from bentgrass (Agrostis stolonifera L.) has been cloned (U.S. Patent Application No. 2005/0235379).

Accordingly, in some embodiments of the present invention, RNAi technology can be used to turn off the expression of one or more endogenous genes involved in the transition from a vegetative to a reproductive growth stage, as set forth above.

The term “antisense” or “antigene” as used herein, refers to any composition containing a nucleotide sequence that is either fully or partially complementary to, and hybridizes with, a specific DNA or RNA sequence. The term “antisense strand” is used in reference to a nucleic acid strand that is fully or substantially complementary to the “sense” strand. Antisense molecules include peptide nucleic acids (PNAs) and may be produced by any method including synthesis, restriction enzyme digestion and/or transcription. Once introduced into a cell, the complementary nucleic acid sequence combines with nucleic acid sequence(s) present in the cell (e.g., as an endogenous or exogenous sequence(s)) to form a duplex thereby preventing or minimizing transcription and/or translation. The designation “negative” is sometimes used in reference to the antisense strand, and “positive” is sometimes used in reference to the sense strand. An antigene sequence can be used to form a hybridization complex at the site of a noncoding region of a gene, thereby modulating expression of the gene (e.g., by enhancing or repressing transcription of the gene).

The term “RNAi” refers to RNA interference. The process involves the introduction of RNA into a cell that inhibits the expression of a gene. Also known as RNA silencing, inhibitory RNA, and RNA inactivation. RNAi as used herein includes double stranded (dsRNA), small interfering RNA (siRNA), small hairpin RNA (or short hairpin RNA) (shRNA) and microRNA (miRNA).

The terms “complementary” or “complementarity,” as used herein, refer to the natural binding of polynucleotides under permissive salt and temperature conditions by base-pairing. For example, the sequence “A-G-T” binds to the complementary sequence “T-C-A.” Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

Different nucleic acids or proteins having homology are referred to herein as “homologues.” The term homologue includes homologous sequences from the same and other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins.

As used herein “sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in a reference sequence segment, i.e., the entire reference sequence or a smaller defined part of the reference sequence.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). In some embodiments, “percent identity” can refer to the percentage of identical amino acids in an amino acid sequence.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., Burlington, Mass.). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

The percent of sequence identity can be determined using the “Best Fit” or “Gap” program of the Sequence Analysis Software Package™ (Version 10; Genetics Computer Group, Inc., Madison, Wis.). “Gap” utilizes the algorithm of Needleman and Wunsch (Needleman and Wunsch, J Mol. Biol. 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, Adv. Appl. Math., 2:482-489, 1981, Smith et al., Nucleic Acids Res. 11:2205-2220, 1983).

Useful methods for determining sequence identity are also disclosed in Guide to Huge Computers (Martin J. Bishop, ed., Academic Press, San Diego (1994)), and Carillo, H., and Lipton, D., (Applied Math 48:1073 (1988)). More particularly, preferred computer programs for determining sequence identity include but are not limited to the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; (Altschul et al., J. Mol. Biol. 215:403-410 (1990)); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

The present invention further provides a transformed plant cell comprising the nucleic acid construct or a multiplicity of different nucleic acid constructs of this invention, in any combination. Furthermore, the elements of the nucleic acid constructs transformed into the plant cell can be in any combination.

A transgenic plant is also provided herein, comprising, consisting essentially of and/or consisting of one or more nucleic acid constructs of this invention. A transgenic plant is additionally provided herein comprising a transformed plant cell of this invention.

Additionally provided herein is a transgenic seed, a transgenic pollen grain and a transgenic ovule of the transgenic plant of this invention. Further provided is a tissue culture of regenerable transgenic cells of the transgenic plant of this invention.

A plant of this invention can be an angiosperm, a gymnosperm, a bryophyte, a fern and a fern ally. In some embodiments the plant is a dicot and in some embodiments, the plant is a monocot. In some embodiments, the plant is perennial and in some embodiments the plant is an annual. In some embodiments, the plant of this invention is a crop plant. Thus, in one embodiment of this invention, a crop of plants is provided, comprising, consisting essentially of or consisting of a plurality of plants of this invention, planted together in an agricultural field.

Nonlimiting examples of a plant of this invention include, turfgrass (e.g., creeping bentgrass, tall fescue, ryegrass, Kentucky Bluegrass), forage grasses (e.g., Medicago trunculata, alfalfa), switchgrass, trees (e.g., orange, lemon, peach, apple, plum, cherry, almond, pecan, poplar, coffee), tobacco, tomato, potato, sugar beet, pea, green bean, lima bean, carrot, celery, cauliflower, broccoli, cabbage, soybean, corn, oil seed crops (e.g., canola, sunflower, rapeseed), cotton, Arabidopsis, pepper, peanut, grape, orchid, rose, dahlia, carnation, cranberry, blueberry, strawberry, lettuce, cassaya, spinach, lettuce, cucumber, zucchini, wheat, maize, rye, rice, flax, oat, barley, sorghum, millet, sugarcane, peanut, beet, potato, legume, sweetpotato, banana, and the like.

Additional embodiments of this invention include methods of producing a transgenic plant and the transgenic plants produced according to the methods described herein.

Thus, in one embodiment, the present invention provides a method of producing a transgenic plant having enhanced tolerance to abiotic stress, comprising:

a) transforming a cell of a plant with one or more (e.g., 2, 3, 4, 5, 6, etc.) nucleic acid constructs of this invention; and b) regenerating the transgenic plant from the transformed plant cell, wherein the plant has enhanced tolerance to abiotic stress as compared with a plant that is not transformed with said nucleic acid construct(s) (i.e., a control plant).

Additionally provided herein is a method of producing a transgenic plant having enhanced biomass production, comprising: a) transforming a cell of a plant one or more (e.g., 2, 3, 4, 5, 6, etc.) nucleic acid constructs of this invention and; and b) regenerating the transgenic plant from the transformed plant cell, wherein the plant has enhanced biomass production as compared with a plant that is not transformed with said nucleic acid construct(s) (i.e., a control plant). In various embodiments, the transgenic plant of this invention can have about 10%, about 20%, about 30%, about 40%, about 50% about 60%, about 70%, about 80%, about 90% or about 100% enhancement in biomass production as compared with the control plant.

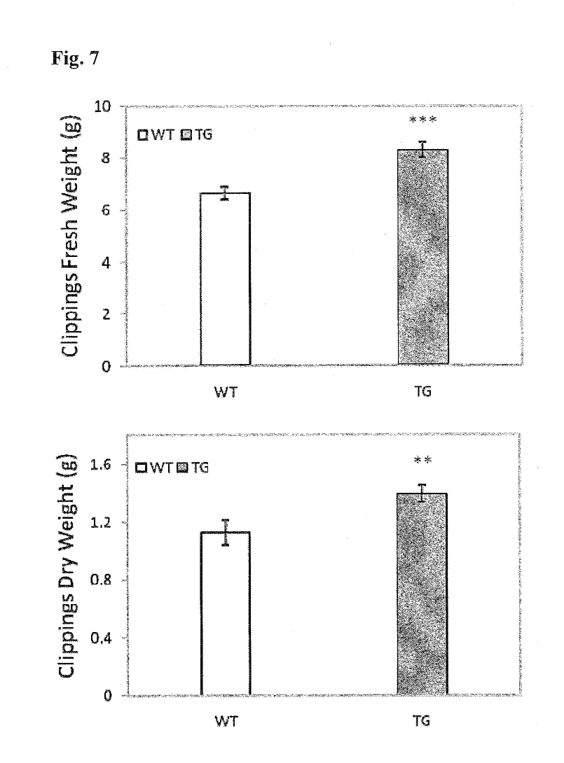

By “enhanced biomass production” is meant that the transgenic plant of this invention is taller, is larger, has greater leaf mass, has greater flower yield, has greater seed production, has a more robust root system, has greater secondary root growth, greater weight of dry clippings and/or greater weight of fresh clippings as compared to a control plant lacking the nucleic acid construct of this invention, maintained under and/or subjected to identical conditions (see, e.g., FIG. 7). Measurement of any or all of these parameters is carried out according to protocols standard in the art.

By ‘enhanced tolerance to biotic and/or abiotic stress” is meant that the transgenic plant of this invention recovers, thrives, survives and/or overcomes a biotic and/or abiotic stress” better than a control plant lacking the nucleic acid construct of this invention, maintained under and/or subjected to identical conditions. In various embodiments, the transgenic plant can have about 10%, about 20%, about 30%, about 40%, about 50% about 60%, about 70%, about 80%, about 90% or about 100% enhancement in tolerance to a biotic stress and/or an abiotic stress as compared with the control plant. In particular embodiments, the transgenic plant of this invention can have about 10%, about 20%, about 30%, about 40%, about 50% about 60%, about 70%, about 80%, about 90% or about 100% enhancement in tolerance to heat stress and/or drought stress as compared with the control plant.

Nonlimiting examples of a biotic and/or an abiotic stress include salt stress, drought stress, heat shock, low temperature, oxidative stress, flowering, phosphate deficiency, pathogen attack, abscisic acid signaling, salicylic acid signaling and any combination thereof.

Measurement of various parameters of the effects of biotic stress and/or abiotic stress is well known in the art and as described herein. For example, transgenic plants and control plants (e.g., wild-type plants) can be exposed to identical salt stress conditions, drought conditions, heat conditions, low temperature conditions, phosphate starvation, pathogen attack, etc. and various parameters of the effect of these types of stress on the different plants are measured to identify enhanced tolerance according to standards methods as are described herein and known in the art.

As noted above, the transgenic plant of this invention can have enhanced tolerance or resistance to attack by a plant pathogen. Nonlimiting examples of the types of pathogens against which a transgenic plant of this invention can have enhanced tolerance or resistance include plant pathogenic fungi, plant pathogenic bacteria, plant pathogenic viruses, plant pathogenic nematodes, plant pathogenic spiroplasmas and mycoplasma-like organisms and plant pathogenic water molds. Nonlimiting examples of a fungal pathogen against which a transgenic plant of this invention can have enhanced tolerance or resistance include Alternaria spp. (e.g. A. longipes, A alternata, A. solani, A. dianthi), Botrytis spp. (e.g., B. cinerea, B. tulipae, B. aclada, B. anthophila, B. elliptica), Cercospora spp. (e.g., C. asparagi, C. brassicicola C. apii), Claviceps spp. (C. purpurea, C. fusiformis), Cladosporium spp. (e.g., C. sphaerospermum, C. fulvum, C. cucumerinum), Fusarium spp. (e.g., F. oxysporum, F. moniliforme, F. solani, F. culmorum, F. graminearum), Helminthosporium spp. (e.g., H. solani, H. oryzae, H. victoriae), Cochliobolus spp., Dreschlera spp., Penicillium spp. (e.g., P. digitatum, P. expansum), Trichoderma spp. (T. viride, T. hamatum), Verticillium spp. (e.g., V. alboatrum, V. dahliae, V. fungicola), Colletotrichum spp. (e.g., C. gloeosporioides, C. lagenarium, C. coccodes, C. orbiculare), Gloeodes spp. (e.g., G. pomigena), Glomerella spp. (e.g., G. cingulata, G. glycines), Gloeosporium solani, Marssonina spp. (e.g., M. populi), Nectria spp. (e.g, N. galligena, N. cinnabarina), Phialophora malorum, Sclerotinia spp. (e.g., S. sclerotiorum, S. trifoliorum, S. homoeocarpa), Magneporthe spp. (e.g., M. grisea, M. salvinii), Rhizoctonia spp. (R. Solani), Mycosphaerella spp. (e.g., M. fijiensis, M. dianthi. M. citri, M. graminicola), Ustilago spp. (e.g., U. maydis), and the like.

Nonlimiting examples of a bacterial pathogen against which a transgenic plant of this invention can have enhanced tolerance or resistance include Pseudomonas spp.(e.g., P. syringae, P. syringae pv. Tabaci, P. marginata), Erwinia spp. (E. carotovora, E. amylovora), Xanthomonas spp., and Agrobacterium spp. (A. tumefaciens, A. rhizogenes), and the like.

Nonlimiting examples of a water mold against which a transgenic plant of this invention can have enhanced tolerance or resistance include Pythium spp. (P. aphanidermatum, P. graminicola, P. ultimatum), Phytophthora spp. (e.g., P. citrophthora, P. infestans, P. cinnamomi, P. megasperma, P. syringae), and the like.

Nonlimiting examples of a nematode against which a transgenic plant of this invention can have increased or enhanced resistance include Xiphenema spp. (X. americanum), Pratylenchus spp. (P. neglectus, P. thornei), Paratylenchus spp. (P. bukowinensis), Criconemella spp. (C. xenoplax, C. curvata; C. ornata), Meloidogyne spp. (M. incognita, M. graminicola, M. arenaria), Helicotylenchus spp. (H. dihystera, H. multicinctus), Rotylenchulus spp., Longidorus spp., Heterodera spp. (H. glycines, H. zeae, H. schachtii), Anguina spp. (A. agrostis, A. tritici), Tylenchulus spp. (T. semipenetrans), and the like.

Nonlimiting examples of a virus against which a transgenic plant of this invention can have enhanced tolerance or resistance include Rhabdovirus, Alfamovirus, Tobomovirus, Luteovirus, Potyvirus, Cucumovirus, Nepovirus, Comoviridae, Sobemovirus, Carlavirus, Ilarvirus, Potexvirus, Caulimovirus, and Geminivirus. Further nonlimiting examples of a virus which a transgenic plant of this invention can have increased or enhanced resistance include tomato spotted wilt virus, tobacco rattle virus, tobacco necrosis virus, tobacco ring spot virus, tomato ring spot virus, cucumber mosaic virus, peanut stump virus, alfalfa mosaic virus, maize streak virus, figwort mosaic virus, tomato golden mosaic virus, tomato mottle virus, tobacco mosaic virus, cauliflower mosaic virus, tomato yellow leaf curl virus, tomato leaf curl virus, potato yellow mosaic virus, African cassaya mosaic virus, Indian cassaya mosaic virus, bean golden mosaic virus, bean dwarf mosaic virus, squash leaf curl virus, cotton leaf curl virus, beet curly top virus, Texas pepper virus, Pepper Huastico virus, alfalfa mosaic virus, bean leaf roll virus, bean yellow mosaic virus, cucumber mosaic virus, pea streak virus, tobacco streak virus, and white clover mosaic virus.

The term “transformation” as used herein refers to the introduction of a heterologous nucleic acid into a cell. Transformation of a cell may be stable or transient. The term “transient transformation” or “transiently transformed” refers to the introduction of one or more heterologous nucleic acids into a cell wherein the heterologous nucleic acid is not heritable from one generation to another.

“Stable transformation” or “stably transformed” refers to the integration of the heterologous nucleic acid into the genome of the plant or incorporation of the heterologous nucleic acid into the cell or cells of the plant (e.g., via a plasmid) such that the heterologous nucleic acid is heritable across repeated generations. Thus, in one embodiment of the present invention a stably transformed plant is produced.

Transient transformation may be detected by, for example, an enzyme-linked immunosorbent assay (ELISA) or Western blot, which can detect the presence of a peptide or polypeptide encoded by one or more transgene introduced into a plant. Stable transformation of a cell can be detected by, for example, a Southern blot hybridization assay of genomic DNA of the cell with nucleic acid sequences which specifically hybridize with a nucleotide sequence of a transgene introduced into a plant. Stable transformation of a cell can be detected by, for example, a Northern blot hybridization assay of RNA of the cell with nucleic acid sequences which specifically hybridize with a nucleotide sequence of a transgene introduced into a plant. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

A nucleotide sequence of this invention can be introduced into a plant cell by any method known to those of skill in the art. Procedures for transforming a wide variety of plant species are well known and routine in the art and described throughout the literature. Such methods include, but are not limited to, transformation via bacterial-mediated nucleic acid delivery (e.g., via Agrobacteria), viral-mediated nucleic acid delivery, silicon carbide or nucleic acid whisker-mediated nucleic acid delivery, liposome mediated nucleic acid delivery, microinjection, microparticle bombardment, electroporation, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

Bacterial mediated nucleic acid delivery includes but is not limited to DNA delivery by Agrobacterium spp. and is described, for example, in Horsch et al. (Science 227:1229 (1985); Ishida et al. (Nature Biotechnol. 14:745 750 (1996); and Fraley et al. (Proc. Natl. Acad. Sci. 80: 4803 (1983)). Transformation by various other bacterial species is described, for example, in Broothaerts et al. (Nature 433:629-633 (2005)).

Physical delivery of nucleotide sequences via microparticle bombardment is also well known and is described, for example, in Sanford et al. (Methods in Enzymology 217:483-509 (1993)) and McCabe et al. (Plant Cell Tiss. Org. Cult. 33:227-236 (1993)).

Another method for physical delivery of nucleic acid to plants is sonication of target cells. This method is described, for example, in Zhang et al. (Bio/Technology 9:996 (1991)). Nanoparticle-mediated transformation is another method for delivery of nucleic acids into plant cells (Radu et al., J. Am. Chem. Soc. 126: 13216-13217 (2004); Torney, et al. Society for In Vitro Biology, Minneapolis, Minn. (2006)). Alternatively, liposome or spheroplast fusion can be used to introduce nucleotide sequences into plants. Examples of the use of liposome or spheroplast fusion are provided, for example, in Deshayes et al. (EMBO J., 4:2731 (1985), and Christou et al. (Proc Natl. Acad. Sci. U.S.A. 84:3962 (1987)). Direct uptake of nucleic acid into protoplasts using CaCl₂ precipitation, polyvinyl alcohol or poly-L-ornithine is described, for example, in Hain et al. (Mol. Gen. Genet. 199:161 (1985)) and Draper et al. (Plant Cell Physiol. 23:451 (1982)). Electroporation of protoplasts and whole cells and tissues is described, for example, in Donn et al. (In Abstracts of VIIth International Congress on Plant Cell and Tissue Culture IAPTC, A2-38, p 53 (1990); D'Halluin et al. (Plant Cell 4:1495-1505 (1992)); Spencer et al. (Plant Mol. Biol. 24:51-61 (1994)) and Fromm et al. (Proc. Natl. Acad. Sci. 82: 5824 (1985)). Polyethylene glycol (PEG) precipitation is described, for example, in Paszkowski et al. (EMBO J. 3:2717 2722 (1984)). Microinjection of plant cell protoplasts or embryogenic callus is described, for example, in Crossway (Mol. Gen. Genetics 202:179-185 (1985)). Silicon carbide whisker methodology is described, for example, in Dunwell et al. (Methods Mol. Biol. 111:375-382 (1999)); Frame et al. (Plant J 6:941-948 (1994)); and Kaeppler et al. (Plant Cell Rep. 9:415-418 (1990)).

In addition to these various methods of introducing nucleotide sequences into plant cells, expression vectors and in vitro culture methods for plant cell or tissue transformation and regeneration of plants are also well known in the art and are available for carrying out the methods of this invention. See, for example, Gruber et al. (“Vectors for Plant Transformation” in Methods in Plant Molecular Biology and Biotechnology, Glick, B. R. and Thompson, J. E., Eds. (CRC Press, Inc., Boca Raton, (1993), pages 89-119).

The term “vector” refers to a composition for transferring, delivering or introducing a nucleic acid (or nucleic acids) into a cell. A vector comprises a nucleic acid comprising the nucleotide sequence to be transferred, delivered or introduced. In some embodiments, a vector of this invention can be a viral vector, which can comprise, e.g., a viral capsid and/or other materials for facilitating entry of the nucleic acid into a cell and/or replication of the nucleic acid of the vector in the cell (e.g., reverse transcriptase or other enzymes which are packaged within the capsid, or as part of the capsid). The viral vector can be an infectious virus particle that delivers nucleic acid into a cell following infection of the cell by the virus particle.

A plant cell of this invention can be transformed by any method known in the art and as described herein and intact plants can be regenerated from these transformed cells using any of a variety of known techniques. Plant regeneration from plant cells, plant tissue culture and/or cultured protoplasts is described, for example, in Evans et al. (Handbook of Plant Cell Cultures, Vol. 1, MacMilan Publishing Co. New York (1983)); and Vasil I. R. (ed.) (Cell Culture and Somatic Cell Genetics of Plants, Acad. Press, Orlando, Vol. I (1984), and Vol. II (1986)). Methods of selecting for transformed transgenic plants, plant cells and/or plant tissue culture are routine in the art and can be employed in the methods of the invention provided herein.

A large variety of plants have been shown to be capable of regeneration from transformed individual cells to obtain transgenic plants. Those of skill in the art can optimize the particular conditions for transformation, selection and regeneration according to these art-known methods. Factors that affect the efficiency of transformation include the species of plant, the tissue infected, composition of the medium for tissue culture, selectable marker coding sequences, the length of any of the steps of the methods described herein, the kinds of vectors, and/or light/dark conditions. Therefore, these and other factors can be varied to determine the optimal transformation protocol for any particular plant species. It is recognized that not every species will react in the same manner to the transformation conditions and may require a slightly different modification of the protocols disclosed herein. However, by altering each of the variables according to methods routine in the art, an optimum protocol can be derived for any plant species.

Accordingly, in one embodiment, a heterologous nucleotide sequence is introduced into a cell of a plant of the present invention by co-cultivation of the cell with Agrobacterium tumefaciens to produce a transgenic plant. In a further embodiment, a heterologous nucleotide sequence is introduced into a cell of a plant of the present invention by direct nucleic acid transfer to produce a transgenic plant.

### EXAMPLES

### Example 1

### Overview of Invention

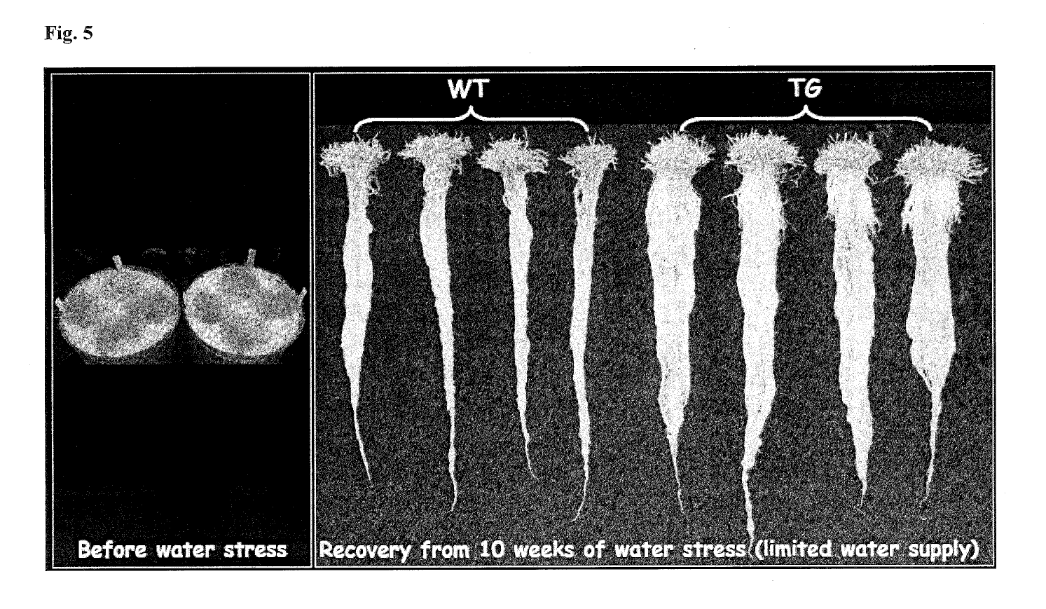

The rice SIZ1 homolog, OsSIZ1, has been cloned and evaluated for the feasibility of its use in turfgrass for improved plant growth and response to abiotic stress. Data described herein have demonstrated that transgenic creeping bentgrass plants overexpressing the OsSIZ1 gene exhibited dramatically enhanced root and shoot growth as well as improved tolerance to drought, heat and cold stresses as well as phosphate starvation (FIGS. 5, 6, 7). This result points to the great potential for a biotechnological approach of genetically engineering plants (e.g., perennials) for enhanced performance.

In one embodiment, this system is implemented in a bioenergy crop, switchgrass (Panicum virgantum L.), in combination with the gene containment strategy described herein to develop an environmentally friendly transgenic switchgrass with enhanced biomass production and improved abiotic stress tolerance. Specifically, transgenic techniques are employed to engineer increased vegetative growth and enhanced tolerance to abiotic stress in switchgrass through overexpression of the rice OsSIZ1 gene. Enhanced biomass production through engineered overexpression of the OsSIZ1 gene in the perennial bioenergy switchgrass will provide an increased amount of renewable source of feed stock for conversion to fuels, reducing total biofuel cost. In addition, transgenic switchgrass plants overexpressing OsSIZ1 exhibit enhanced tolerance to abiotic stresses, such as drought, cold, heat and phosphate starvation. This will greatly improve plant adaptation to adverse environmental conditions and enhance plant growth and development for stable biomass production.

Using an RNA interference approach, the switchgrass FLO/LFY homolog, a gene controlling transition from vegetative to reproductive growth of plant, is down-regulated, achieving total sterility of transgenic plants for the purpose of transgene containment. The implementation of total sterility in transgenic plants will not only promote plant vegetative growth, contributing to enhancing plant biomass production, but also provides an effective way to prevent transgene escape through pollen and seeds and makes it possible for the engineered switchgrass with enhanced abiotic stress tolerance to be used in the field. The results obtained will lead to potentially new cultivars for commercialization. This molecular strategy can also be used to engineer controlled total sterility in turfgrass for seed production under contained conditions, which could be developed as a second generation of genetically modified plants for commercialization.

Energy security and climate change imperatives require large-scale substitution of the decreasing reserves of fossil fuels. The need on a global scale for energy crops as renewable fuels and alternative sources of farm income is of great importance to current ecological and economic issues. Fast growing warm season perennial grasses have been identified as ideal candidates for biomass fuel production due to their high net energy yield per hectare and low cost of production. In particular, the C₄ grass switchgrass (Panicum virgantum L.) holds considerable promise as a biomass fuel. Switchgrass is mainly planted for land conservation, and utilized for forage and hay (Moser and Vogel, 1995). It has the following advantages as a bioenergy crop: moderate to high productivity, stand longevity, high moisture and nutrient use efficiency, low cost of production and adaptability to most agricultural regions in North America. Switchgrass has an energy output to input ratio of approximately 20:1, and typically can produce 175.5 MBtu of energy per 10 ton of biomass from land that is often of marginal crop producing value. The United States Department of Energy designated switchgrass as a potential bioenergy feedstock because of its wide adaptability and high yields on marginal lands (Vogel, 1996). Switchgrass use as a bioenergy feedstock, in addition to providing energy, might reduce net carbon gas emissions, improve soil and water quality, increase native wildlife habitat, and increase farm revenues (McLaughlin and Walsh, 1998; McLaughlin et al., 2002).

Transgene escape through pollen dispersion raises valid ecological concerns regarding commercialization of transgenic perennials. In aspects of this invention, total sterility can be incorporated into the final product with engineered desirable traits. This strategy provides an effective system for gene containment that will guarantee safe use of genetically modified plants of the perennial switchgrass.

### Example 2

### Materials and Methods

Plasmid construction and bacterial strains. The binary vector, pSB11 (Hiei et al. 1994), was used to prepare the OsSIZ1-expression chimeric gene construct, pUbi-OsSIZ1/35S-bar (FIG. 1) for turfgrass transformation. The construct contains the corn ubiquitin promoter driving the rice SUMO E3 ligase gene OsSIZ1 that is linked to the cauliflower mosaic virus 35S (CaMV 35S) promoter driving the bar gene for herbicide resistance as the selectable marker.

The ORF of OsSIZ1 gene was amplified from cDNA of rice spike tissue by using the primer pair OsSIZ1F (5′-GAGATCTGAGTAGGGAGGCGGGCGAACC-3′, SEQ ID NO:6) and OsSIZ1R (5′-GAG ATCTCCAGACGACCGATAACCCCACCTCAG-3′, SEQ ID NO:7), and cloned into a pGEM-T-Easy vector (Promega, Madison, Wis., U.S.A.). After sequencing, a 2777 bp BglII (New England Biolabs, Beverly, Mass., USA) fragment was released from the cloning vector, blunted with a large Klenow fragment (New England Biolabs, Beverly, Mass., USA), and cloned into the blunted SacI-BamHI (New England Biolabs, Beverly, Mass., USA) fragment of pSBUbi-35S::bar. The sense orientation clone was confirmed by PCR with the primer pair OsSIZ1R and Ubi-int-SEQ1 (5′-ACTTGGATGATGGCATATGCAGCAG-3′, SEQ ID NO:8). The construct was delivered into Agrobacterium tumefaciens strain, LBA4404, by electroporation for plant transformation.

Plant materials and transformation. Creeping bentgrass (Agrostis stolonifera L.) cultivar, cv. Penn A-4, supplied by HybriGene (Hubbard, Oreg., USA), was used for transformation in this study. Transgenic creeping bentgrass lines stably expressing OsSIZ1 were produced using Agrobacterium-mediated transformation of embryogenic callus initiated from mature seeds essentially as described (Luo et al. 2004). The regenerated transgenic plants from tissue culture were transferred in commercial potting mixture soil (Fafard 3-B Mix, Fafard Inc., Anderson, S.C., USA) or pure silica sand and maintained in the greenhouse under 16 hour photoperiods with supplemental lighting at 27° C. in the light and 25° C. in the dark.

Southern and Northern analysis of transgenic plants. Plant genomic DNA was isolated from 1 g of young leaves using the cetyltrimethyl ammonium bromide (CTAB) method (Luo et al. (1995). T-DNA inserted into the host genome of the transgenic plants was confirmed by PCR amplification of a 0.44 kb fragment of the selectable marker gene, bar and a 638 bp fragment of the OsSIZ1 gene. The two primers used for bar amplification were BarF (5′-GTCTGCACCATCGTCAACCACTAC-3′, SEQ ID NO:9) and BarR (5′-GTCCAGCTGCCAGAAACCCAC-3′, SEQ ID NO:10), while those for OsSIZ1amplification were OsSIZ1-q-PCRF (5′-GTGAAGATCAGCGATGCCAAGTGTG-3′, SEQ ID NO:11) and OsSIZ1-q-PCRR (5′-CTCTGCAGTGTCTCCACCTCCGAG-3′, SEQ ID NO:12). For Southern analysis of plant genomic DNA, twenty micrograms of DNA were digested with HindIII (New England Biolabs, Beverly, Mass., USA). Digested DNA was fractionated through a 0.7% (w/v) agarose gel and blotted on to a Hybond-N⁺ filter (GE Healthcare Bio-Sciences Corp., Piscataway, N.J., USA).

Total RNA was extracted with Trizol reagent (Invitrogen, Carlsbad, Calif., USA) from 0.1 g of the young leaves of transgenic and wild-type plants, and treated with RNase free DNase I (Invitrogen, Carlsbad, Calif., USA). Upon electrophoresis in agarose gel, denatured total RNA (20 μg per lane) was blotted on to a Hybond-N⁺ filter (GE Healthcare Bio-Sciences Corp., Piscataway, N.J., USA), following a standard protocol (Sambrook, Fritsch & Maniatis 1989).

For DNA hybridization, the 440 bp fragment of bar gene amplified from plasmid DNA with primers as described above was used as probe, while for RNA hybridization, the 1.0 kb fragment of OsSIZ1 gene amplified from plasmid DNA with primers as described above was used as probe. Probes were labeled with [α-³²P]dCTP using a Prime-It II Random Primer Labeling Kit (Stratagene, La Jolla, Calif., USA). DNA blot was probed in Church buffer at 65° C. RNA blot was probed in Church buffer at 68° C. and exposed on phosphor at RT overnight and scanned using a Typhoon 9400 scanner (GE Healthcare Bio-Sciences Corp., Piscataway, N.J., USA).

OsSIZ1 Expression Pattern and Expression Level in Rice and Transgenic Turfgass. The expression pattern and level of OsSIZ1 were examined as described (Li et al., 2009). Briefly, RT-PCR primers designed from a rice tubulin-α (X91808) and Actin 1 (GenBank® Accession No. NM_{—}001057621) EST were used as a positive amplification control and as a quantitative standard to assess relative gene expression. Template cDNA of leaf, root, flower, and panicles from different development stages was synthesized from 2.5 μg of DNase I (Invitrogen, Carlsbad, Calif., U.S.A.) treated total RNA using the SuperScript III First Strand Synthesis System for RT-PCR (Invitrogen, Carlsbad, Calif., U.S.A.). First strand cDNAs were diluted with nuclease-free water and aliquots of the cDNA sample were amplified using gene-specific primers. PCR reactions were performed in a 25 μl volume containing 0.2 μM of each primer, 1 U of Platinum Taq DNA polymerase (Invitrogen, Carlsbad, Calif., U.S.A.), 0.2 mM dNTPs, 1.5 mM MgCl₂, 0.4 μM of each primers and 1×Taq polymerase reaction buffer, 4 μl of cDNA sample. PCR was performed as follows: denaturation at 95° C. for 60 s; 20-25 cycles of 94° C. for 30 s, 62° C. for 30 s, 72° C. for 40 s; and extension at 72° C. for 120 s. PCR products were fractionated on a 2% (w/v) agarose gel, stained with SYBR Green I (Invitrogen), and photographed using FUJIFILM Science Lab 99 Image Gauge system. The relative intensities of the bands in each lane were quantified by imaging the gel with the Image Gauge Ver. 3.2 software (Fuji Photo Film, Tokyo, Japan). Target gene expression was quantitative relative to the control amplification (tubulin-1 and actin 1) in the same lane of the gel.

Preparation of Plant Materials for Stress Test. to Produce Abundant Plant Materials for use in evaluating plant response to stress, two events of transgenic creeping bentgrass plants (TG1 and TG2) and wild-type (WT) controls were clonally propagated from stolons and grown in cone-tainers (4.0 cm×20.3 cm, Dillen Products, Middlefield, OH, USA; five individual stolons per cone-tamer) and pots (15 cm×10.5 cm, Dillen Products, Middlefield, OH, USA; 50 individual stolons per pot) using pure silica sand. Under growth room climate, the plants were developed at a fourteen hour photoperiod for 6 weeks. Illumination in the growth room was 350-450 μmol m⁻²s⁻¹ photosynthetically active radiation at canopy height provided by AgroSun© Gold 1000W sodium/halide lamps (Maryland Hydroponics, Laurel, Md., USA). Temperatures were maintained at 25° C. in the light and 17° C. in the dark and relative humidity was 30%/60% (light/dark). The plants were watered every other day with 200 ppm of water soluble fertilizer (20-10-20 Peat-Lite Special, the Scotts Company, OH, USA). During this period, the grass shoots were clipped weekly to achieve uniform plant growth.

Roots and leaves of the plants from the Dillen cone-tainers were then trimmed to the same size and repotted with fifteen individual stolons in new cone-tainers containing 225±1 g pure sand, and arranged in a pentagon shape. The trimmed plants were also repotted in Elite 1200 Pot (27.9 cm×24.6 cm, ITML, Middlefield, OH, USA). Sixty stolons of wild type and each transgenic event were arranged in a hexagon shape with 2 replicates in the same Elite 1200 Pot.

The grasses in the Dillen cone-tainers or the Elite 1200 Pots were arranged randomly and the above-ground parts were clipped to 2 cm or 5 cm length for both transgenic and wild type before stress treatments. After 10 weeks development in the growth room under the same conditions mentioned above, the plants in the Dillen cone-tainers and the Elite pots were conducted to different stresses.

Heat stress. The ten replicates of both WT and two transgenic events in the Dillen cone-tainers and the four replicates in the Elite pots were transferred to the growth chamber (Conviron, Controlled Environments Inc., Pembina, ND U.S.A), maintained under the same temperature, photoperiod, and light density mentioned above for one week to allow them to adjust to the environment before heat treatments.

The temperatures for heat stress experiment were maintained at 35° C. under the light and 30° C. in the dark for 7 days and then at 40° C. under the light and 35° C. in the dark for 7 days, and relative humidity was 60%-80%. Heat-stressed plants were well-watered every two days with 200 ppm of fertilizer (20-10-20), and the cone-tainers and pots were sunk in the 200 ppm of fertilizer solution (around 4 cm from bottom).

The materials were harvested for proline test in the 9^{th} day and 14^{th} treatment

Turf quality was determined by the integral of the relative water content (RWC), leaf chlorophyll content (Li et al, 2009), and visually turf quality (density, color and uniformity).

Pathogen Test A Sclerotinia homoeocarpa inoculum was prepared as previously described (Chakraborty et al. 2006). Briefly, the pathogen isolate was grown on PDA for 1 week at 21° C. under constant fluorescent light. Inoculum was prepared by autoclaving 15 g of oat (Avena sativa L.) seeds twice with 20 ml of Difco potato dextrose broth (PDB) in flasks. Four 4-mm-diameter culture plugs were excised from the growing edge of each fungal colony and transferred to the oat seed medium and allowed to grow for 3 week at room temperature, with 12 h of light and daily shaking to prevent clumping of the seeds.

The grass in the cone-tainers and Elite pots was inoculated with approximately 0.2 g of inoculum by even distribution on the top of the grass. Inoculated plants were randomly placed in a growth chamber with >90% relative humidity, with a temperature range from 22 to 27° C., which is optimal for maximum pathogenicity, and maintained in a diurnal cycle of 14 h light and 10 h dark. Four replicates of each transgenic event or wild type were used. The disease symptom severity was visually estimated at 3, 5 and 7 days post-inoculation using the Horsfall/Barrett scale (Horsfall et al, 1945).

Drought Tolerance Test. The 4 replicates of WT and two transgenic events in the Dillen cone-tainers and Elite pots were maintained for five to six weeks. The volumetric water content (VWC) of pure sand was measured by using a TDR 200 Soil Moisture Meter (Spectr m Technologies, Inc, Plainfield, Ill. USA). The plants were maintained for 10 weeks under growth room condition described above. Drought-stressed plants were provided with limited water every five days and VWC ranged from 1 to 5%. After 10 weeks treatment, the weight of root and leaf of the plants was investigated. During drought tolerance test, the leaves of the plants were sampled every two weeks for leaf electrolyte leakage, relative water content (RWC) and proline content.

Phosphate Starvation Test. The transgenic and wild type plants developed in the pure sand were washed and trimmed carefully to the same size (4.5 cm of leaf length, 1.5 cm of root length), and repotted in the cone-tainers containing pure sand, and watered with the basal nutrition (containing 1×MS micronutrients, 1/10× macronutrients without KH₂PO₄) without or with various amounts of KH₂PO₄ every day (for cone-tainers) or every three days (for Elite1200 pots). The watering of plants was performed until free drainage occurred from the bottom of cone-tainers and pots.

Measurement of mineral contents. (See also Li et al. “Heterologous expression of Arabidopsis H⁺-pyrophosphatase enhances salt tolerance in transgenic creeping bentgrass (Agrostis stolonifera L.)” Plant, Cell and Environment 33:272-289 (2009), the entire contents of which are incorporated by reference herein.)

For salt-stress treatment of plants, the water soluble fertilizer (20-10-20 Peat-Lite Special, the Scotts Company, Ohio, USA) solution is supplemented with NaCl to a final concentration of 0, 100, 200, or 300 mM for application. Plant leaf and root samples are collected to determine their standard minerals and soluble chloride. The amounts of Na⁺, K⁺, Mg²⁺, total phosphorus, and soluble chloride in creeping bentgrass leaves and roots of wild-type controls and transgenic plants (TG1) is measured. All shoots with stems (approximately 3 cm above silica sand) of the creeping bentgrass plants are rinsed in Millipore water for 30 seconds, and used to measure the mineral contents. The roots are rinsed in Millipore water to eliminate the silica sand and used to determine the minerals and soluble chloride contents. Leaves and roots are dried for 48 h at 80° C., and the dry weights are measured. The minerals and soluble chloride contents in leaves and roots are determined using Spectro ARCOS ICP (Spectro, Mahwah, N.J., USA) in Clemson University Agricultural Service Laboratory following protocols by Haynes (1980) and Plank (1992).

Measurement of leaf relative water content. Leaf relative water content (RWC) is estimated using the following formula: RWC=[(FW−DW)/(TW−DW)]×100%, where FW is fresh weight, DW is dry weight, and TW is turgid weight. The leaves from both the transgenic and wild-type plants are harvested and immediately weighted (FW). They are then cut into pieces and immersed in Millipore water at 4° C. for 16 h. After measuring the turgid weight (TW), the leaves are dried in an oven at 80° C. for 24 h and weighed (DW).

Measurement of leaf electrolyte leakage. Leaf electrolyte leakage (EL) is measured to evaluate cell membrane stability. For EL analysis, fresh leaf segments (0.2-0.5 g) from each sample are incubated in 20 ml Millipore water at 4° C. for 16 h. The conductance of the incubation solution is measured as the initial level of EL (Ci) using a conductance meter (AB30, Fisher Scientific, Suwanee, Ga., USA). This measurement estimates the amount of the ions released from cells under normal or salt stressed conditions. Leaf tissues in the incubation solution are then killed by autoclaving for 30 min. The conductance of the incubation solution with killed tissues (Cmax) is determined following 24 h incubation on a shaker. This measurement reflects the amount of the ions released from plant cells before and after heat killing (i.e., the total amount of ions contained in the leaf samples). Relative EL is calculated as (Ci/cmax)×100.

Proline content determination. Proline content is determined essentially after Bates et al. (1973) with minor modifications. Briefly, proline is extracted from 100 mg of plant leaves by grinding in 2 ml of 3% sulfosalicylic acid. Two hundred micro liters of extract is reacted with 200 μl of acid ninhydrin and 200 μA of glacial acetic acid for 60 min at 100° C. An ice bath is used to terminate the reaction. The reaction mixture is extracted with 1000 μl of toluene and vortexed. Absorbance of the toluene layer is read at 520 nm in a Thermo Spectronic BioMate 3 (Thermo Electron Corp., Waltham, Mass., USA) and proline concentration is determined from a standard curve and calculated on a fresh weight basis as follows: [μg proline/ml×(μl toluene/μl sample)/(g sample/10)]/115.5 μg/μmol=μmol proline/g of fresh weight material.

Chlorophyll measurement. Changes of leaf chlorophyll contents in wild-type controls and transgenic plants (TG1) subjected to salt stress (100 mM NaCl) are determined over a period of 12 days upon NaCl treatment. One hundred milligrams of fresh leaf tissue is cut into small pieces with scissors. The pigment is extracted by grinding for 5 minutes in 10 ml of 85% acetone in a mortar and pestle. The homogenate is transferred into a 15-ml Falcon tube and spun at 3,000×g for 15 minutes. The supernatant is then transferred into a new 15-ml Falcon tube and made up to volume with 85% acetone. The optical density (absorbance) of the extract is measured at both 663 nm and 644 nm with the Thermo Spectronic BioMate 3 (Thermo Electron Corp., Waltham, Mass., USA). The concentration of chlorophyll a and b, in milligram per gram of fresh weight (FW) tissue, is calculated after Amon (1949) and Koski (1950) using the following formula:

Milligram chlorophyll a/g FW=1.07(OD₆₆₃)−0.094(OD₆₄₄)

Milligram chlorophyll b/g FW=1.77(OD₆₄₄)−0.280(OD₆₆₃)

Indole-3-acetic acid extraction and measurement by high-performance liquid chromatography. Indole-3-acetic acid (IAA) is isolated principally after Bruns et al. (1997) with modifications. Fifteen grams of fresh tissue from wild-type and transgenic plants is ground in fine powder in liquid nitrogen with a mortar and pestle, and extracted with 50 ml of methanol containing butylhydroxytoluene (1 mg/ml) for 120 min under continuous shaking in the dark. The supernatant is collected and filtered through a 0.22 μm nylon membrane filter (OSMONICS, Minnetonka, Minn., USA). The filtrate is evaporated in a vacuum rotary concentrator (room temperature) up to the aqueous phase, and then passed again through a 0.22 μm nylon membrane filter. The concentrated filtrate is adjusted to pH 3.5 with glacial acetic acid (around 3 μl/ml filtrate) and applied to a Sep-Pak C-18 cartridge (500 mg, Waters, Milford, Mass., USA), which is pre-equilibrated with 2 ml of methanol followed by 2 ml of 50 mM acetic acid. The cartridge is washed with 2 ml of 50 mM acetic acid followed by 2 ml of water. The IAA is eluted with 2 ml of methanol, and concentrated in a vacuum rotary concentrator (room temperature) to 200 which is further purified by passing through a 0.22 μm Cellulose Acetate Spin-X® Centrifuge Tube Filter (Corning Inc., Corning, N.Y., USA).

IAA from plant tissue extraction is quantified by high-performance liquid chromatography (HPLC) according to Li et al. (2007). A YMC-Pack-Pro C18 column (250 mm×4.6 mm, S-5 μm, 12 nm, YMC Inc, Milford, Mass., USA) is connected to the LC-10AT HPLC system (Shimadzu, Kyoto, Japan) with a SPD-20A/AV detector (280 nm). For each sample, twenty to forty microliters of the methanolic extract is injected and eluted with 1% (v/v) acetic acid/acetonitrile/(75/25, v/v) at a flow rate of 0.8 ml/min. The levels of free IAA in samples are quantified using a calibration curve of the standards (0, 5, 25, 100, and 500 ppm of IAA). The standards are treated by passing through the cartridge and spin column prior to HPLC. Samples are measured four times and the standard error is calculated.

Measurement of H₂O₂. Samples of 200 mg WT and transgenic plant leaves ground in liquid N₂ are homogenized in 1 ml 10% (v/v) H₃PO₄. The supernatant is used for the determination of H₂O₂ and lipid hydroperoxide by the methods of Wolff (1994). The reaction mixture for H₂O₂ analysis contains 100 mM xylenol orange, 250 mM ammonium ferrous sulphate, 100 mM sorbitol, 25 mM H₂SO₄ and 50 ml extract in a total volume of 1 ml.

The following mixture is used for the measurement of lipid hydroperoxide concentration: 100 mM xylenol orange, 250 mM ammonium ferrous sulphate, 90% methanol (HPLC grade), 4 mM butylated hydroxytoluene, 25 mM H₂SO₄ and 50 ml extract in a total volume of 1 ml. For both compounds, calibration is performed using H₂O₂.

Salicylic acid (SA) extraction and measurement by HPLC. Shoots from plants are grown in pure sand under conditions described herein and harvested and frozen in liquid nitrogen. Tissue (0.2 g fresh weight, without roots) is extracted in 4 mL of methanol for 24 h at 4° C. and then in a solution of 2.4 mL of water plus 2 mL of chloroform with 40 μL of 5 mM 3,4,5-trimethoxy-trans-cinamic acid (internal standard) for 24 h at 4° C. Supernatants are dried by speed vacuum. The residue is resuspended in 0.4 mL of water:methanol (1:1, v/v), and SA is quantified by HPLC at 25° C. using a Nova-Pak C-18 column with a flow rate of 1 mL min⁻¹ over 22 min using a methanol gradient (solvent A, water and 1% formate; and solvent B, 100% methanol and 1% formate) of 10% to 40% B (10 min), 40% to 50% B (5 min), 50% to 100% B (2.5 min), 100% to 40% B (2.5 min), 40% to 10% B (1 min), and 10% B (1 min).

Phosphate starvation test and measurement of mineral contents. Transgenic (TG) and wild-type (WT) plants developed in the pure sand were washed and trimmed carefully in same size (4.5 cm of leaf length, 1.5 cm of root length), and repotted in the cone-tainers with pure sand, and nurtured with the basal nutrition (1× MS micronutrients, 1/10× macronutrients without or with various amounts of KH₂PO₄) daily (for cone-tainers) or every three days (for Elite1200 pots).

Ten weeks after treatment, plant leaf and root samples were collected to determine their standard minerals. The amounts of Na⁺, K⁺, Mg²⁺ and total phosphorus in leaves and roots of WT controls and TG plants were measured. Both shoots and roots were rinsed in Millipore (Billerica, Mass., USA) water briefly, and then dried for 48 h at 80 C. After measuring the dry weights (DWs), the minerals in leaves and roots were determined using Spectro ARCOS ICP (Spectro, Mahwah, N.J., USA) in Clemson University Agricultural Service Laboratory following protocols by Haynes (1980) and Plank (1992).

### Example 3

### Results

Overexpression of SIZ1 results in enhanced drought tolerance in transgenic creeping bentgrass plants. Replicates of transgenic and wild-type plants were asexually propagated from stolons in Elite 1200 Pot with pure silica sand. The plants were maintained in a growth room and trimmed weekly for ten weeks to achieve uniform growth. To examine how the SIZ1-expressing transgenic plants perform in response to drought stress, the restricted water supply was applied to both wild-type and transgenic plants for a period of 10 weeks. Results from plants grown in the Elite 1200 Pot subjected to ten weeks of exposure to drought conditions (1%-5% volumetric water content of sand) and then to two weeks of water saturated conditions (10%-21% volumetric water content of sand) indicated that although transgenic and wild-type plants were both affected in root development, the transgenic plants exhibited less growth inhibition and faster recovery upon sufficient nutrition and water supply. Examination of plant root development revealed that SIZ1-overexpressing transgenic plants developed a more robust root system than the wild-type controls under drought condition. With two weeks of sufficient nutrition and water supply, the new roots in the transgenic plants were abundantly growing, whereas those of wild-type controls were poorly developed (FIG. 5).

As demonstrated, transgenic plants seemed to perform better than wild-type controls, exhibiting greater root growth under stress conditions. To evaluate whether overexpressed SIZ1 impacts overall plant growth and development under drought condition, experiments were conducted to compare plant root biomass in transgenic and wild-type plants. The results indicated that the biomass of root by fresh and dry weights in transgenic plants was significantly greater than that in wild-type controls.

Overexpression of SIZ1 results in enhanced thermotolerance in transgenic creeping bentgrass plants. To examine the performance of the SIZJ-overexpressing transgenic plants under heat stress compared to wild-type controls, the transgenic and wild-type plants were grown in cone-tainers or in Elite 1200 Pots with pure silica sand and maintained in the growth room for six or ten weeks, respectively, to achieve uniform growth and plants were trimmed weekly. Ten cone-tainers of each plants and two Elite 1200 Pots were then sunk in the 200 ppm of fertilizer solution in a container (around 4 cm from bottom), and treated in the growth chamber under heat stress conditions for two weeks as described herein.

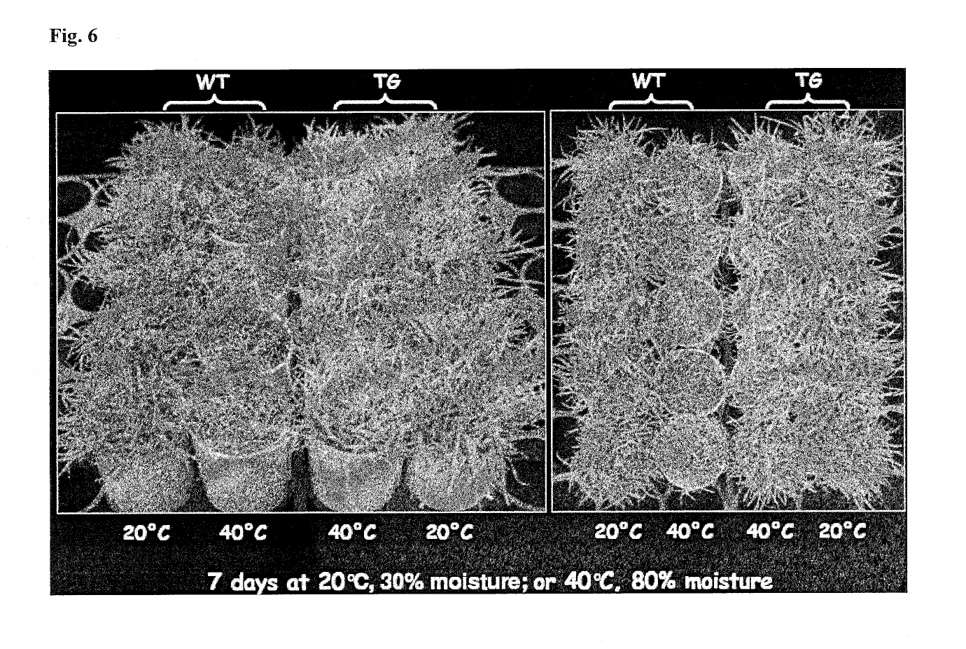

As demonstrated in FIG. 6, transgenic plants performed better than wild-type controls in both of cone-tainers and Elite 1200 Pot under heat stress conditions.

Results from plants grown in cone-tainers subjected to two weeks of exposure to heat stress indicated that although transgenic and wild-type plants were both affected in shoot development, the transgenic plants exhibited less growth inhibition and tissue damage than wild type plants. Similar results were also observed for plants grown in Elite 1200 Pots under heat stress treatment for two weeks. Transgenic plants displayed enhanced thermotolerance under stress conditions and faster recovery under the maintaining conditions for two weeks. In the cone-tainers test, two weeks of high temperature treatment was lethal to wild-type plants, whereas under the same conditions, transgenic plants had less damage and were able to recover from the damage. Similar results were obtained in the Elite 1200 Pot test. Under the same conditions, most of the wild type plants could not recover from lethiferous damages, however, transgenic plants were able to recover from the lighter damage in two weeks.

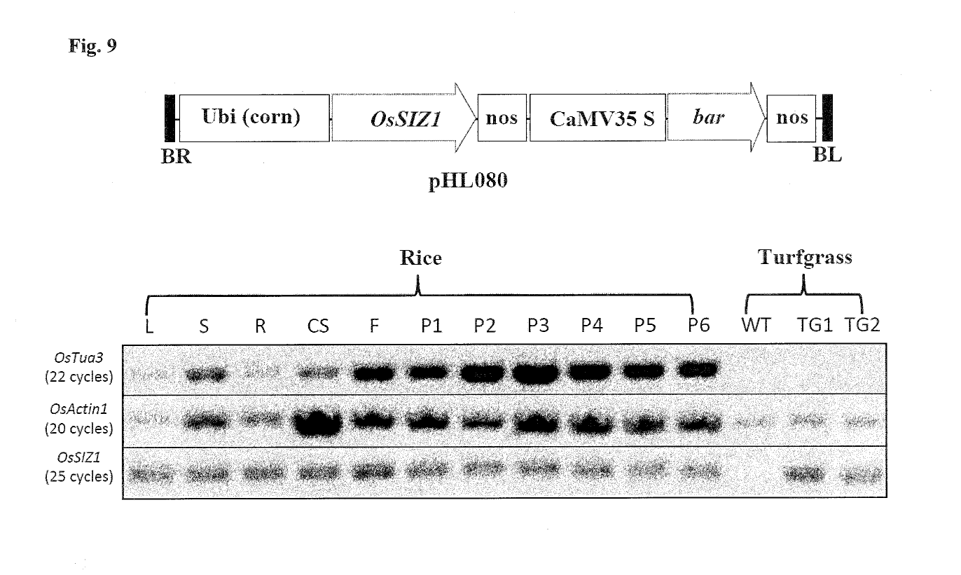

Expression pattern and level of OsSIZ1 in rice and transgenic creeping bentgrass plants. The expression pattern and level of OsSIZ1 were analyzed following procedures as previously described (Li, et al., 2009). Briefly, rice α-tubulin and actin genes were used as references and as quantitative standard to assess relative gene expression of OsSIZ1. First strand cDNAs were diluted with nuclease-free water and aliquots of the cDNA samples were amplified using gene-specific primers. PCR reactions were performed in a 25 μl volume containing 0.2 μM of each primer, 1 U of Platinum Taq DNA polymerase, 0.2 mM dNTPs, 1.5 mM MgCl₂, and 1×Taq polymerase reaction buffer, 4 μl of cDNA sample. PCR was performed as follows: denaturation at 95° C. for 60 s; 20-25 cycles of 94° C. for 30 s, 62° C. for 30 s, 72° C. for 40 s; and extension at 72° C. for 120 s. PCR products were fractionated on a 2% (w/v) agarose gel, stained with SYBR Green I (Invitrogen), and photographed using FUJIFILM Science Lab 99 Image Gauge system. Target gene expression was quantitative relative to the control amplification (α-tubulin and actin 1) in the same lane of the gel. Tissues from greenhouse-grown rice and transgenic creeping bentgrass plants were harvested to determine the expression of OsSIZ1 by RT-PCR. Vegetative (leaf and root) and flora tissues (flower and panicles) were sampled on multiple dates throughout the development and maturation (heading) stage in the greenhouse. Root samples were obtained from greenhouse-grown potted rice and were 10-day old, white roots. OsSIZ1 is constitutively expressed in all rice tissues (FIG. 9). Its expression was detected only in the transgenic creeping bentgrass plants (TG1 and TG2, FIG. 9), but not in the non-transgenic wild-type controls (WT, FIG. 9).

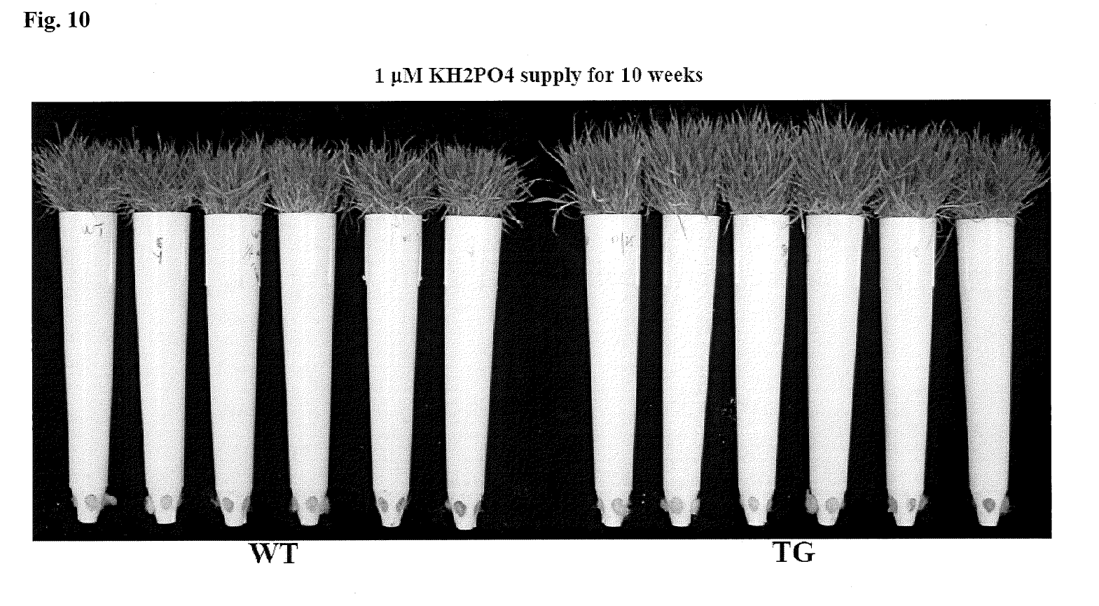

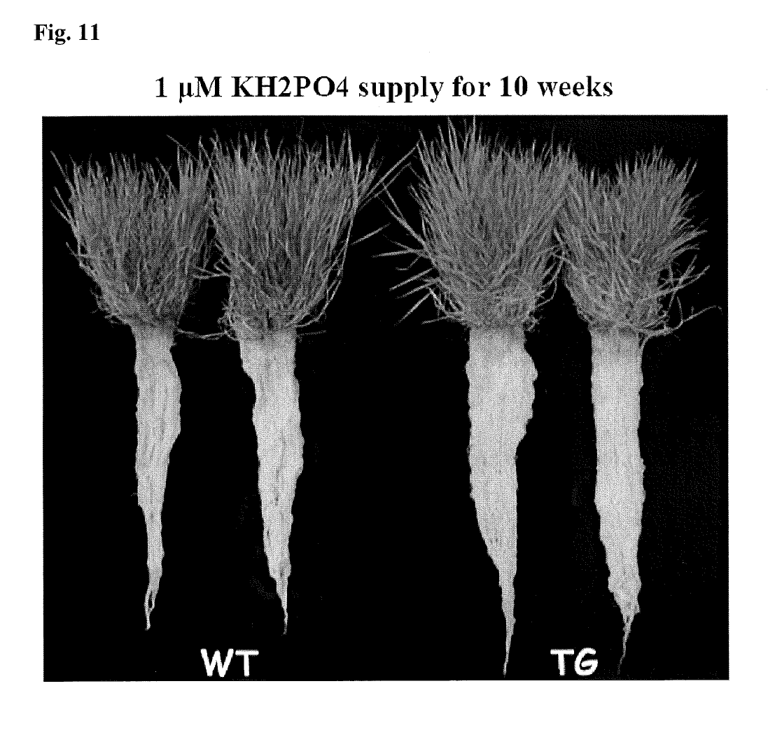

Overexpression of OsSIZ1 in transgenic creeping bentgrass led to enhanced uptake of phosphate and potassium improved plant performance under phosphate starvation. To examine how the OsSIZ1-overexpressing transgenic (TG) plants perform in ion uptake compared to wild-type (WT) controls, leaf and root phosphate and potassium contents were measured in plants treated with different concentrations of KH₂PO₄ (1 and 10 μM). As demonstrated in FIGS. 10-11, when grown under 1 μM KH₂PO₄ application for ten weeks, WT plants exhibited typical phosphate deficiency symptom with a significantly inhibited growth, whereas TG plants showed much better performance. Under normal growth conditions, the total phosphate and K⁺ levels in plant tissues (leaves and roots) of both TG and WT plants were similar. However, when subjected to low phosphate conditions (1 and 10 μM KH₂PO₄), the total phosphate and K⁺ levels started to decline in both TG and WT plants. This decline was more pronounced in the root, and both TG and WT plants exhibited much lower K⁺ levels than normal growth conditions.

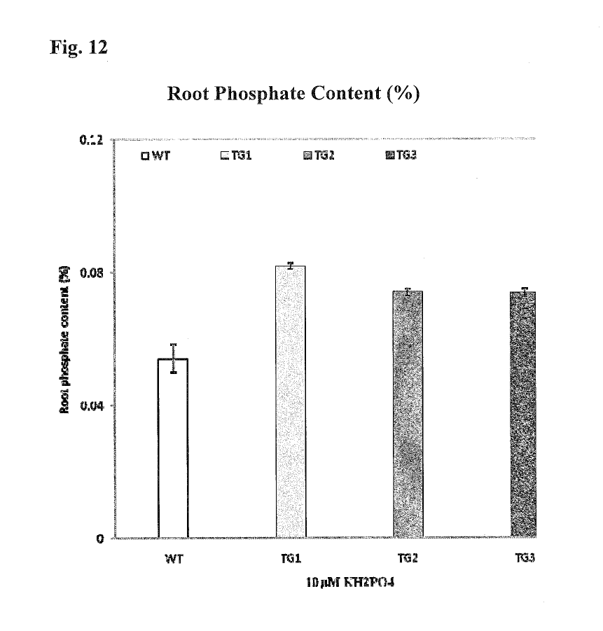

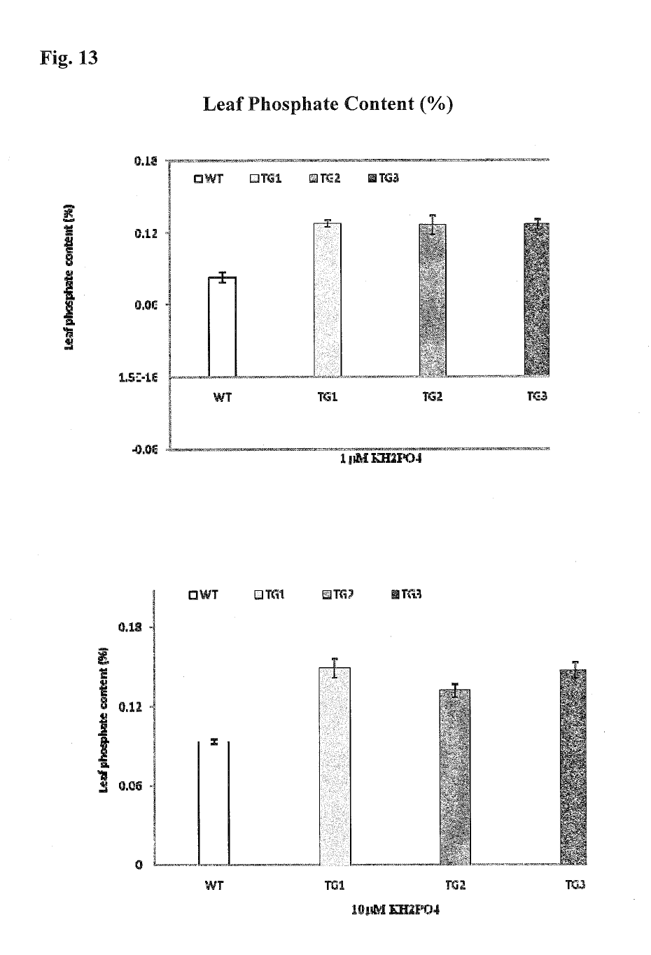

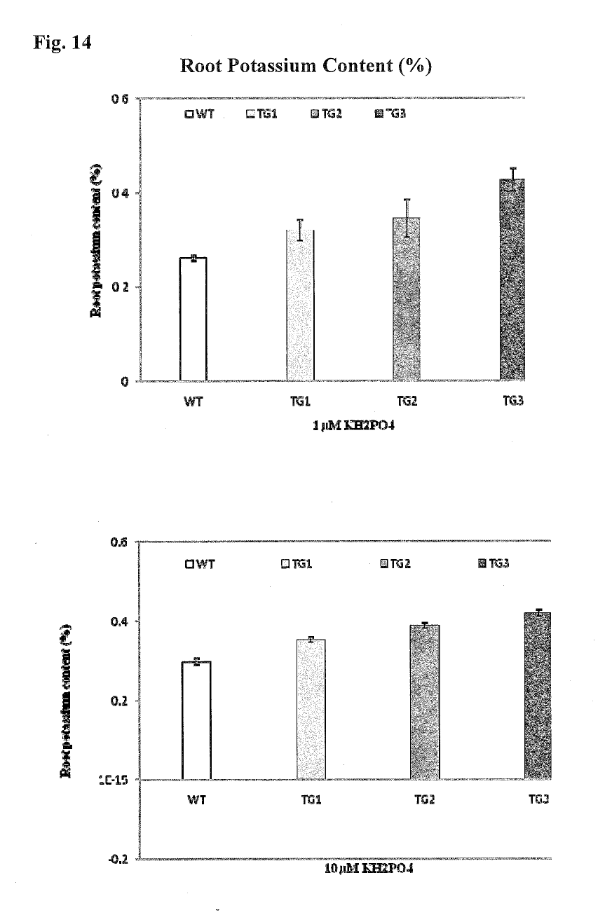

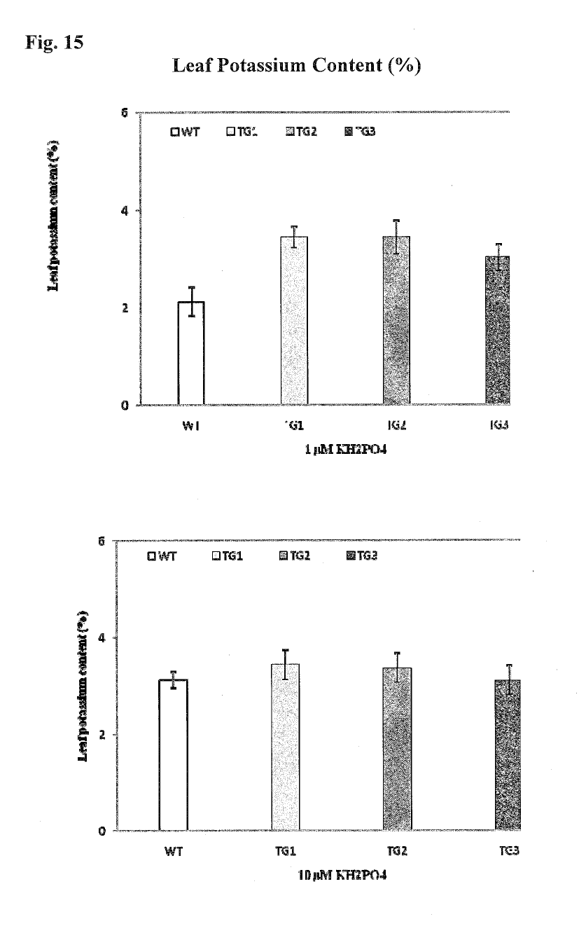

The impact of low phosphate on minerals levels in both WT and TG creeping bentgrass plants was evaluated. As shown in FIG. 12, TG plants accumulated more phosphorus in roots than WT controls, attaining about 30-41% higher under 10 μM KH₂PO₄ supply conditions. Although leaf total phosphorus content in WT and TG plants both declined with decreasing concentrations of KH₂PO₄, the decrease was more rapid and significant in WT plants (FIG. 13). Similar results were obtained for plant root potassium content with both WT and TG plants when subjected to low concentrations of KH₂PO₄ (1 and 10 μM) treatment. TG plants showed significantly higher potassium content than WT controls (FIG. 14). Interestingly, compared to WT controls, TG plants exhibited significantly increased leaf potassium content when 1 μM of KH₂PO₄ was supplied; however, with 10 μM of KH₂PO₄ supply, there were no significant differences in leaf potassium content between WT and TG plants (FIG. 15).

The foregoing is illustrative of the present invention, and is not to be construed as limiting thereof. The invention is defined by the following claims, with equivalents of the claims to be included therein.

All publications, patent applications, patents and other references cited herein are incorporated by reference in their entireties for the teachings relevant to the sentence and/or paragraph in which the reference is presented.

### REFERENCES

Catala R, Ouyang J, Abreu I A, Hu Y, Seo H, Zhang X, Chua N—H (2007) The Arabidopsis E3 SUMO ligase SIZ1 regulates plant growth and drought responses. Plant Cell 19:2952-2966.
Conti L, Price G, O'Donnell E, Schwessinger B, Dominy P, Sadanandom A (2008) Small ubiquitin-like modifier proteases OVERLY TOLERANT TO SALT1 and -2 regulate salt stress responses in Arabidopsis. Plant Cell 20:2894-2908.
Geiss-Friedlander R, Melchior (2007) Concepts in sumoylation: a decade on. Mol Cell Biol 8:947-956.
Hanania U, Furman-Matarasso N, Ron M, Avni A (1999) Isolation of a novel SUMO protein from tomato that suppresses EIX-induced cell death. Plant J 19:53-541.
Jin J B, Jin Y H, Lee J, Miura K, Yoo C Y, Kim W-Y, Oosten M V, Hyun Y, Somers D E, Lee I, Yun D-J, Bressan R A, Hasegawa P M (2008) The SUMO E3 ligase, AtSIZ1, regulates flowering by controlling a salicylic acid-mediated floral promotion pathway and through affects on FLC chromatin structure. Plant J 53:530-540.
Kurepa J, Walker J M, Smalle J, Gosink M M, Davis S J, Durham T L, Sung D-Y, Vierstra R D (2003) The small ubiquitin-like modifier (SUMO) protein modification system in Arabidopsis: Accumulation of SUMO1 and -2 conjugates is increased by stress. J Biol Chem 278:6862-6872.
Lee J, Nam J, Park H C, Na G, Miura K, Jin J B, Yoo C Y, Baek D, Kim D H, Jeong J C, Kim D, Lee S Y, Salt D E, Mengiste T, Gong Q, Ma S, Bohnert H J, Kwak S-S, Bressan R A, Hasegawa P M, Yun D-J (2006) Salicylic acid-mediated innate immunity in Arabidopsis is regulated by SIZ1 SUMO E3 ligase. Plant J 49:79-90.
Lois L M, Lima C D, Chua N-H (2003) Small Ubiquitin-Like Modifier Modulates Abscisic Acid Signaling in Arabidopsis. Plant Cell 15:1347-1359.
McLaughlin S B and Walsh M E (1998) Evaluating environmental consequences of producing herbaceous crops for bioenergy. Biomass Bioenergy 14:317-324.
McLaughlin S B, De La Torre Ugarte Jr D G, Garten C T, Lynd L R, Sanderson M A, Tolbert V R, Wolf D D (2002) High value renewable energy from prairie grasses. Environ Sci Technol 36:2122-2129.
Miura K, Jin J B, Lee J, Yoo C Y, Stirm V, Miura T, Ashworth E N, Bressan R A, Yun D-J, Hasegawa P M (2007) SIZ1-mediated sumoylation of ICE1 controls CBF3/DREB1A expression and freezing tolerance in Arabidopsis. Plant Cell 19:1403-1414.
Miura K, Lee J, Jin J B, Yoo C Y, Miura T, Hasegawa P M (2009) Sumoylation of ABI5 by the Arabidopsis SUMO E3 ligase SIZ1 negatively regulates abscisic acid signaling. Proc Natl Acad Sci USA 106:5418-5423.
Miura K, Rus A, Sharkhuu A, Yokoi S, Karthikeyan A S, Raghothama K G, Baek D, Koo Y D, Jin J B, Bressan R A, Yun D-J, Hasegawa P M (2005) The Arabidopsis SUMO E3 ligase SIZ1 controls phosphate deficiency responses. Proc Natl Acad Sci USA 102:7760-7765.
Moser L E, Vogel K P (1995) Switchgrass, big bluestem, and indiangrass. In: Forages: An introduction to grassland agriculture, Barnes R F et al (eds.). Iowa State Univ Press, Ames, I A. pp. 409-420.
Murtas G, Reeves P H, Fu Y-F, Bancroft I, Dean C, Coupland G (2003) A nuclear protease required for flowering-time regulation in Arabidopsis reduces the abundance of SMALL UBIQUITIN-RELATED MODIFIER conjugates. Plant Cell 15:2308-2319.
Saracco S A, Miller M J, Kurepa J, Vierstra R D (2007) Genetic analysis of SUMOylation in Arabidopsis: conjugation of SUMO1 and SUMO2 to nuclear proteins is essential. Plant Physiol 145:119-134.
Vogel K P (1996). Energy production from forages (or American agriculture-back to the future). J Soil Water Consery 51:137-139.
Yoo C Y, Miura K, Jin J B, Lee J, Park H C, Salt D E, Yun D-J, Bressan R A, Hasegawa P M (2006) SIZ1 small ubiquitin-like modifier E3 ligase facilitates basal thermotolerance in Arabidopsis independent of salicylic acid. Plant Physiol 142:1548-1558.

That which is claimed is:

1. A nucleic acid construct comprising, in the following order from 5′ to 3′:
a) a first promoter;
b) a nucleotide sequence encoding small ubiquitin-related modifier (SUMO) E3 ligase or an active fragment thereof operably associated with the promoter of (a);
c) a first termination sequence;
d) a second promoter;
e) a nucleotide sequence encoding a selectable marker operably associated with the promoter of (d); and
f) a second termination sequence.

2. The nucleic acid construct of claim 1, comprising in the following order from 5′ to 3′:
a) a corn ubiquitin promoter;
b) a nucleotide sequence encoding rice SUMO E3 ligase;
c) a first nos sequence;
d) a CaMV 35S promoter;
e) a nucleotide sequence encoding phosphinothricin acetyltransferase (bar); and
f) a second nos sequence.

3. A transformed plant cell comprising the nucleic acid construct of claim 1.

4. A transformed plant cell comprising the nucleic acid construct of claim 2.

5. A transgenic plant comprising the nucleic acid construct of claim 1.

6. A transgenic plant comprising the nucleic acid construct of claim 2.

7. A transgenic plant comprising the transformed plant cell of claim 3.

8. A transgenic plant comprising the transformed plant cell of claim 4.

9. A transgenic seed from the transgenic plant of claim 7.

10. A transgenic seed from the transgenic plant of claim 8.

11. A method of producing a transgenic plant having enhanced biomass production, comprising:
a) transforming a cell of a plant with the nucleic acid construct of claim 1; and
b) regenerating the transgenic plant from the transformed plant cell, wherein the plant has enhanced biomass production as compared with a control plant that is not transformed with said nucleic acid construct.

12. A method of producing a transgenic plant having enhanced tolerance to biotic and/or abiotic stress, comprising:
a) transforming a cell of a plant with the nucleic acid construct of claim 1; and
b) regenerating the transgenic plant from the transformed plant cell, wherein the plant has enhanced tolerance to biotic and/or abiotic stress as compared with a control plant that is not transformed with said nucleic acid construct.

13. The method of claim 12, wherein the stress is selected from the group consisting of:
a) salt stress;
b) drought stress;
c) heat stress;
d) oxidative stress;
e) low temperature;
f) flowering;
g) phosphate deficiency;
h) pathogen attack;
i) abscisic acid signaling;
j) salicylic acid signaling and
k) any combination of (a)-(j) above.

14. The method of claim 12, wherein the stress is drought stress.

15. The method of claim 11, wherein the transgenic plant has at least 10% enhancement in biomass production as compared with the control plant.

16. The method of claim 12, wherein the transgenic plant has at least 10% enhancement in tolerance to biotic and/or abiotic stress as compared with the control plant.

17. A transgenic plant produced by the method of claim 11.

18. A transgenic plant produced by the method of claim 12.

19. A crop comprising a plurality of plants according to claim 17, planted together in an agricultural field.

20. A crop comprising a plurality of plants according to claim 18, planted together in an agricultural field.
